(19) United States
(12) Patent Application Publication
(10) Pub. No.: US 2011/0165603 A1
(43) Pub. Date: Jul. 7, 2011

(54) SMALL MOLECULE FLUORESCENT SENSORS FOR DETECTION OF POST-TRANSLATIONALMODIFICATIONS AND PROTEIN INTERACTIONS IN BIOASSAYS

(75) Inventors: Frauke H. Rininsland, Santa Fe, NM (US); Shannon Keeley Wittenburg, Santa Fe, NM (US); Wendy L. Weatherford, Santa Fe, NM (US)

(73) Assignee: Gyrasol Technologies Inc., Santa Fe (NM)

(21) Appl. No.: 13/061,412

(22) PCT Filed: Aug. 26, 2009
(86) PCT No.: PCT/US09/55097

§ 371 Date: Feb. 28, 2011

Related U.S. Application Data
(60) Provisional application No. 61/190,129, filed on Aug. 26, 2008.
(60) Provisional application No. 61/146,053, filed on Jan. 21, 2009.

(51) Int. Cl. C12Q 1/48; C07K 7/06; C07K 7/08; C07F 9/40; C07J 51/00; C07H 23/00; C07F 9/655; C07F 5/02; C12Q 1/42; C12Q 1/37

(52) U.S. Cl. 435/15; 530/329; 530/326; 530/327; 530/328; 558/155; 536/17.1; 552/506; 536/121; 549/220; 548/405; 435/21; 435/23

(57) ABSTRACT

The present invention relates to novel compounds which are capable as acting as fluorescent sensors or which are precursors for these and for the use of these for the assay of biological processes such as posttranslational modifications of biological molecules such as phosphorylation, de-phosphorylation, proteolytic cleavage, phosphodiesterase mediated hydrolysis of cyclic nucleotides, methylation, acetylation of proteins peptides, DNA, lipids and the detection of biomolecule interactions (e.g., protein-protein interactions). A small molecule sensor is described which can associate to phosphorylated biological targets via metal ion—phosphate association. The association event can be monitored as fluorescence quench, sensitized emission, fluorescence polarization or a combination thereof. The sensor is useful for determining enzyme

### CROSS-REFERENCE TO RELATED APPLICATIONS

This application claims priority to U.S. Provisional Application for Patent No. 61/190,129, filed Aug. 26, 2008, and 61/146,053, filed Jan. 21, 2009, the entire contents of both of which are specifically incorporated herein by reference.

### BACKGROUND OF THE INVENTION

Post-translational modification (PTM) of proteins (e.g., enzymes) plays many import roles in cellular physiology. PTM is typically accomplished by enzymes that recognize and modify the structure of a protein substrate to yield a modified protein product. Some enzymes that belong to the class of post-translational modifying proteins include, for example, protein kinases, phosphatases, proteases, methylases, acetylases, phosphodiesterases, and lipases. Protein kinases are enzymes that use adenosine triphosphate (ATP) as a phosphate donor and transfer a phosphate group to a specific protein substrate. Protein phosphatases catalyze the reverse process, namely the removal of a phosphate from a substrate. Methylases and acetylases transfer methyl or acetyl groups to substrates, respectively. Proteases, phosphodiesterases and lipases cleave their respective substrates.

PTM of proteins is the main regulator of cellular signaling and can confer or abolish activity to an enzyme or otherwise alter the structure of a protein so that it gains the ability to bind to another protein or to disassociate from it. Aberrant regulation of PTM is implicated in various diseases such as cancer, diabetes, hypertension, and inflammatory diseases. Increasing efforts are being placed on the discovery of drugs that can be used to modulate the activity of enzymes that are inappropriately activated and alter the structure of proteins following expression. This has created the demand for techniques that can easily measure the activity of enzymes.

Distinct cellular phenotypes are the result of differential activation of cellular signaling pathways. More than 500 kinases are involved in regulating signal transduction by activating or de-activating their molecular targets by virtue of phosphorylation. In addition to the enzymes discussed above, phosphodiesterases, enzymes that hydrolyze cyclic nucleotides, are involved in the regulation of signaling pathways. Aberrant protein activity within a pathway is often the result of genetic variations and is implicated in various diseases such as cancer and diabetes. In addition, signaling pathways can be manipulated by viruses and other infectious agents in a manner that is conducive to the infectious agent's survival and propagation. Targeting the effector response of disease as opposed to the source has led to the successful treatment of infection using drugs known to inhibit the activity of an aberrantly regulated kinase (see, for example, Wei et al., Antimicrob Agents Chemother. 2007 December; 51(12): 4321 1328, Ruhland et al. Exp. Parasitol. 2009 May; 122 (1); 28-36, and Stantchev et al., Virus Res 2007 February; 123(2); 178-189.) Thus, cellular signaling pathway profiling is an approach that can further the understanding of the mechanisms and treatment of disease and can have a high potential impact in areas of genetics, infection and immunology.

Assays for detecting the activity of kinase enzymes are known. Radioactive assays have historically been used for this purpose (see for example U.S. Pat. Nos. 5,538,858; 4,568,649; 5,665,562 and 5,989,854). However, because of the cost and health concerns associated with handling of radioactive materials, fluorescence-based methods were developed to replace radioactive methods. In order to be compatible with the robotic screening of thousands to millions of chemical compounds in a process called “high throughput screening” (HTS), homogeneous assays are most desirable. Some non-radioactive, homogeneous assays are based on fluorescence resonance energy transfer (FRET) and fluorescence polarization (FP) (see, for example U.S. Pat. Nos, 6,287,774 and 7,306,928). In FRET methods, a light-absorbing dye, capable of light emission (donor fluorophore), is combined with another fluorophore (acceptor fluorophore) that has spectral overlap with the first fluorophore. When the two dyes are brought into close proximity to each other, excitation of the donor fluorophore results in energy being transferred to the second, acceptor fluorophore and consequently, the emission from the first, donor fluorophore is decreased. If the acceptor fluorophore is capable of emitting light, the light absorbed form the donor fluorophore can be re-emitted by the acceptor fluorophore in a process called sensitized emission. Fluorescence quench of a donor fluorophore can also be achieved by the process of electron or charge transfer in which an electron is transferred to the acceptor. This process does not require spectral overlap between the acceptor and the donor fluorophore and thus any acceptor fluorophore that is within the UV-visible range can be used for energy transfer based assays.

In FP, the readout relies on a measurable change in fluorescence polarization of a fluorophore-labeled substrate, which is achieved by its binding to a molecule of greater size. The increase in size decreases the speed of molecular rotation of the substrate and increases the amount of polarized light emitted from the fluorophore-labeled substrate. The amount of polarized light is calculated as the ratio of two separate emission events monitored on the parallel and perpendicular plane.

Assays can be constructed using FRET techniques where specific binding events can be utilized to bring two fluorophores into close proximity. For example, assays that are based on the affinity of paramagnetic metal ions to phosphates present on substrates labeled with a fluorophore have been described. In these assays, metal ions are coupled to metal ion coordinating groups. In one invention, (U.S. Pat. No. 7,306,928) the sensor consists of a complexed paramagnetic ion, which, upon association to a fluorophore-labeled substrate, extinguishes the fluorescence of the substrate. In another invention (U.S. Pat. No. 6,699,655) the metal ion is complexed to the surface of a microsphere. Upon binding to a phosphorylated and fluorophore-labeled substrate the change in FP of the substrate is monitored. A similar microsphere-based approach is disclosed in US published patent application 2007/0238143 in which the microsphere is co-coated with a conjugated fluorescent polymer, which undergoes superquenching upon association to a fluorophore-labeled and phosphorylated substrate.

Drawbacks of the FRET technique include the requirement of two fluorophores with spectral overlap, and negative assay interactions, such as non-specific interaction of the sensor with the dye-labeled substrate. Additionally, assays are generally of a “turn off” type, unless sensitized emission is recorded. Assays based on FP are popular due to the fact that they generate a “turn on” signal. However, drawbacks of FP include the requirement for expensive equipment capable of monitoring FP, non-specific signals associated with incorporation of small molecular weight fluorophores into large detergent micelles and limitations of the size of substrate that can be detected.

Metal ions described for use in kinase/phosphatase, protease and phosphodiesterase assays are the paramagnetic metal iron (U.S. Pat. No. 7,306,928) that quenches fluorescence of a dye-labeled substrate in FRET assays or change the molecular rotation of a fluorophore-labeled substrate in FP assays (U.S. Pat. No. 6,699,65). Additionally, gallium chloride (US Pub. No. 2007/0238143) has been used in a kinase/phosphatase platform in FRET assays where one of the fluorescent species was a conducting, conjugated polymer capable of fluorescence superquenching. The metal ion zirconyl chloride has recently been described as another useful metal ion that associates to phosphates with larger specificity than iron or gallium when complexed to phosphonate groups present on polystyrene microspheres (Feng et al., Mol & Cell. Proteom. 6:1656-1665, 2007). Rather than associating the metal ion to a solid support, small molecule fluorescent sensors chemically modified to contain a phosphonate group have been employed to detect the presence of metal ions in solutions (US 2007-0049761 A1).

Currently available kinase assays that monitor the accumulation of ADP or depletion of ATP (4, 5) are not adaptable to cellular lysates because they cannot discriminate between the activity of the target of interest and the many other enzymatic events within a cell that convert ATP to ADP. Similarly, assays that rely on secondary readout enzymes, such as proteases suffer from non-specific cleavage by intracellular enzymes.

Commercially available platforms that are adaptable to cellular lysates and are homogeneous include Invitrogen's (Carlsbad, Calif.) TR-FRET based GFP-fusion protein assays. Since the GFP fusion-protein substrate must be transfected into a cell these assays are not suitable for monitoring differences in a physiologically relevant context. Other cell-based platforms, such as ALPHA Screen-based Surefire (Perkin Elmer, Waltham, Mass.), RayBio (Norcross Ga.), or Bioplex (Hercules, Calif.) rely on antibodies to capture phosphoproteins and are not able to quantify the actual activity that is conferred to a phosphorylated target within a signaling pathway.

While FRET and FP platforms are popular for monitoring of single enzyme activity in biochemical assays, none of the platforms are conducive to multiplexed applications. The main disadvantage of FRET assays is the necessary spectral overlap between the donor and acceptor fluorophores (15). Therefore, sensors based on FRET can monitor the modulation of only one fluor and are not adaptable to multiplexed applications. While FP-based assays monitor the modulation of only one fluor, for most instruments the readout requires specialized instrument configurations depending on the type of fluor used. The configurations cannot be simultaneously applied, thus making FP-based applications restricted to the readout of only one fluor.

There remains a need in the art for assays to detect PTM, particularly of enzymes involved in signal transduction. Further, there remains a need in the art for assays capable of simultaneously determining the activity levels of multiple enzymes involved in the same or different signal transduction pathways. These needs and others are met by the present invention.

### BRIEF SUMMARY OF THE INVENTION

In accordance with the present invention, there is provided a method and composition of matter for assaying the activity of an enzyme based on modulation of fluorescence.

In one embodiment, the present invention provides a composition comprising a substrate complexed to a metal ion. Substrates of the invention may comprise a fluorescent moiety attached to a body portion. Substrates may also comprise one or more phosphoryl groups that may be attached to the body portion. Typically, one or more metal ions may be complexed to the one or more phosphoryl groups. Generally speaking, at least one phosphoryl group and at least one fluorescent moiety are positioned on the body such that at least some fluorescence from the fluorescent moiety is quenched by the metal ion that is complexed to the substrate. In one embodiment, the metal ion is complexed to the substrate via an interaction with at least one phosphoryl group. In one embodiment, the metal ion may be zirconium.

In some embodiments, the body portion of a substrate of the invention may comprise a peptide. Any peptide that may be acted upon by an enzyme of interest may be used in the practice of the invention. Peptides may be derived from larger sequences (i.e., proteins) that are acted on by an enzyme of interest. In some embodiments, peptides comprise an amino acid sequence that is recognized by an enzyme of interest. An example of a peptide that may be used in the practice of the invention is a peptide comprising the sequence LRRASLG (SEQ ID NO:1, also known as kemptide). This sequence is recognized by protein kinase A (PKA). In the presence of PKA and ATP this peptide is phosphorylated at the serine residue. Another example of a peptide sequence that may be used in the practice of the invention is a peptide comprising the sequence KVEKIGEGTYGVVYK (SEQ ID NO:2) a sequence that is recognized by the protein kinase Fyn. Those of skill in the art are aware of numerous peptide sequences that are acted on by enzymes of interest and peptides comprising such sequences may be used in the practice of the invention. Other suitable peptides include, but are not limited to: GRPRTSSFAEG (SEQ ID NO:3) a sequence that is recognized by Akt1/PKB, p70S6, and MAPKAPK1; GRTGRRNSI (SEQ ID NO:4) a sequence that is recognized by PKA; ARKRERTYSFGHHA (SEQ ID NO:5) a sequence that is recognized by AKT/PKB and rac; KRELVEPLTPSGEAPNQALLR (SEQ ID NO:6) a sequence that is recognized by ERK1/2 and p44/p42MAPK; RRAAEELDSRAGSPQL (SEQ ID NO:7) a sequence that is recognized by GSK3; PLARTLSVAGLPGKK (SEQ ID NO:8) a sequence that is recognized by camKII; KQAEAVTSPR (SEQ ID NO:9) a sequence that is recognized by PKC; RFARKGSLRQKNV (SEQ ID NO:10) a sequence that is recognized by PKC and PKA; APRTPGGRR (SEQ ID NO:11) a sequence that is recognized by p44MAPK, p42MAPK, ERK1/2, and p38α, GEEPLYWSFPAKKK (SEQ ID NO:12) a sequence that is recognized by Blk, Btk, ckit, IGF-1R, vEGF-R1, and src.

The body portion of a substrate of the invention may be of any chemical composition that is recognized by an enzyme of interest. Body portions of the substrates of the invention may comprise, for example, lipids, nucleotides (e.g., cyclic nucleotides such as cAMP and cGMP), oligonucleotides, and/or carbohydrates. Examples of suitable lipids include, but are not limited to, sphingosine, diacyl glycerol, phosphatidyl-myo-inositol, lipids involved in cellular signaling, phosphatidylinositol phosphates (PIPs), prostaglandins, steroid hormones such as estrogen, testosterone and cortisol, and oxysterols such as 25-hydroxy-cholesterol. Suitable carbohydrates include, but are not limited to, myo-inositol, glucose, fructose, and sorbitol.

Substrates of the invention may comprise one or more fluorescent moieties. Any fluorescent moiety known to those skilled in the art may be used. Suitable examples of fluorescent moieties that may be used in the practice of the invention include, but are not limited to, TAMRA dyes, BODIPY dyes, fluorescein, CHROMEO dyes, DyLight dyes, cyanine dyes, R-phycoerythrin (PE), fluorescein, lissamine rhodamine B, Texas Red, allophycocyanin (APC), Cy3.5, Cy 5.5, and Cy7.

In one embodiment, the present invention provides a method of detecting a kinase enzyme in a sample. Such a method may comprise contacting the sample with a substrate for the kinase enzyme. Typically, as described above, a substrate for use in the methods of the invention may comprise a fluorescent moiety. Contacting the sample with the substrate is typically performed under condition in which the enzyme of interest is known to be active. Such conditions may include pH, monovalent cation (e.g., Na+) concentration, divalent cation (e.g., Mg²⁺) concentration, etc. Determination of such conditions is routine in the art. After contacting the sample with substrate, the reaction is allowed to proceed for a desired period of time. The reaction mixture may then be contacted with a sensor of the invention. Sensors of the invention typically comprise a metal ion, for example, zirconium. Fluorescence may then be detected from the substrate, wherein a decrease in fluorescence indicates the presence of the kinase enzyme.

Any type of kinase enzyme may be assayed using the methods of the invention by simply varying the make up of the substrate such that the substrate comprises a recognition site for the enzyme of interest. This is typically accomplished by varying the body portion of the substrate. A body portion of a substrate may comprise a peptide, a lipid and/or a carbohydrate depending on the enzyme of interest to be assayed. In one embodiment, the body portion comprises a peptide, for example, a peptide comprising an amino acid sequence selected from the group consisting of SEQ ID NOs:1-12. In another embodiment, the body portion comprises a lipid, for example, a lipid selected from a group consisting of sphingosine, diacyl glycerol, phosphatidyl-myo-inositol, phosphatidylinositol phosphates (PIPs), prostaglandins, steroid hormones such as estrogen, testosterone and cortisol, and oxysterols such as 25-hydroxy-cholesterol. In another embodiment, the body portion comprises a carbohydrate, for example, a carbohydrate selected from the group consisting of myo-inositol, myo-inositol, glucose, fructose, and sorbitol. Substrates for use in assaying kinase enzymes typically comprise a fluorescent moiety, for example, a fluorescent moiety selected from a group consisting of TAMRA dyes, BODIPY dyes, fluorescein, CHROMEO dyes, DyLight dyes, cyanine dyes, R-phycoerythrin (PE), fluorescein, lissamine rhodamine B, Texas Red, allophycocyanin (APC), Cy3.5, Cy 5.5, and Cy7.

In one embodiment, the present invention provides a method of detecting a phosphatase enzyme in a sample. Such a method may comprise contacting the sample with a substrate for the phosphatase enzyme. Typically, as described above, a substrate for use in the methods of the invention may comprise a fluorescent moiety. Contacting the sample with the substrate is typically performed under condition in which the enzyme of interest is known to be active. Such conditions may include pH, monovalent cation (e.g., Na+) concentration, divalent cation (e.g., Mg²⁺) concentration, etc. Determination of such conditions is routine in the art. After contacting the sample with substrate, the reaction is allowed to proceed for a desired period of time. The reaction mixture may then be contacted with a sensor of the invention. Sensors of the invention typically comprise a metal ion, for example, zirconium. Fluorescence may then be detected from the substrate, wherein an increase in fluorescence indicates the presence of the phosphatase enzyme.

Any type of phosphatase enzyme may be assayed using the methods of the invention by simply varying the make up of the substrate such that the substrate comprises a recognition site for the enzyme of interest. This is typically accomplished by varying the body portion of the substrate. A body portion of a substrate may comprise a peptide, a lipid, a nucleotide, and/or a carbohydrate depending on the enzyme of interest to be assayed. In one embodiment, the body portion comprises a peptide, for example, a peptide comprising GLGF(pY)MAYG (SEQ ID NO:13), which acts a substrate for the phosphatase PTP-1B. In another embodiment, the body portion comprises a lipid, for example, a lipid selected from a group consisting of sphingosine phosphate, diacyl glycerol phosphate, phosphatidyl-myo-inositol phosphate. In another embodiment, the body portion comprises a lipid, for example, a lipid selected from a group consisting of sphingosine, diacyl glycerol, phosphatidyl-myo-inositol, phosphatidylinositol phosphates (PIPs), prostaglandins, steroid hormones such as estrogen, testosterone and cortisol, and oxysterols such as 25-hydroxy-cholesterol. In another embodiment, the body portion comprises a carbohydrate, for example, a carbohydrate selected from the group consisting of myo-inositol, myo-inositol, glucose, fructose, and sorbitol. Substrates for use in assaying phosphatase enzymes typically comprise a fluorescent moiety, for example, a fluorescent moiety selected from a group consisting of TAMRA dyes, BODIPY dyes, fluorescein, CHROMEO dyes, DyLight dyes, cyanine dyes, R-phycoerythrin (PE), fluorescein, lissamine rhodamine B, Texas Red, allophycocyanin (APC), Cy3.5, Cy 5.5, and Cy7.

In one embodiment, the present invention provides a method of detecting a protease enzyme in a sample. Such a method may comprise contacting the sample with a substrate for the protease enzyme. Typically, as described above, a substrate for use in the methods of the invention may comprise a fluorescent moiety and a phosphoryl group separated by a peptide sequence comprising the recognition site for the protease enzyme. The fluorescent moiety and the phosphoryl group are typically situated such that in the presence of a sensor of the invention, fluorescence is quenched. When the peptide is cleaved by the action of the protease, the phosphoryl group and the fluorescent moiety become separated and the fluorescent moiety is no longer quenched. Contacting the sample with the substrate is typically performed under condition in which the enzyme of interest is known to be active. Such conditions may include pH, monovalent cation (e.g., Na+) concentration, divalent cation (e.g., Mg²⁺) concentration, etc. Determination of such conditions is routine in the art. After contacting the sample with substrate, the reaction is allowed to proceed for a desired period of time. The reaction mixture may then be contacted with a sensor of the invention. Sensors of the invention typically comprise a metal ion, for example, zirconium. Fluorescence may then be detected from the substrate, wherein an increase in fluorescence indicates the presence of the protease enzyme.

### BRIEF DESCRIPTION OF THE DRAWINGS

FIG. 1 shows various schematic representations of assays of the invention. FIG. 1A is a schematic of a fluorescence quench assay of the invention. FIG. 1B is a schematic representation of a FP assay of the invention using a fluorescent small molecular sensor. FIG. 1C shows the use of the present invention for assaying phosphodiesterase activity. FIG. 1D shows an embodiment of the invention using intramolecular quenching. FIG. 1E shows an embodiment invention using intermolecular quenching.

FIG. 2A is a plot of relative fluorescence units (RFU) versus % phosphopeptide showing a linear dose response using TAMRA-labeled peptide. FIG. 2B is a plot of relative fluorescence units (RFU) versus % phosphopeptide showing a linear dose response using Fluorescein-labeled peptide.

FIG. 3A is a line graph plot of relative fluorescence units (RFU) versus concentration of kinase showing the results of an assay for protein kinase A. FIG. 3B is a line graph plot of relative fluorescence units (RFU) versus concentration of kinase showing the results of an assay for Fyn kinase. FIG. 3C is a line graph plot of relative fluorescence units (RFU) versus concentration of kinase showing the results of an assay for sphingosine kinase I. FIG. 3D is a line graph plot of relative fluorescence units (RFU) versus concentration of kinase showing the results of an assay for phosphoinositide-3-kinase. FIG. 3E is a line graph plot of % phospholipid versus concentration of kinase showing the results of an assay for phosphoinositide-3-kinase.

FIG. 4A is a plot of RFU versus time at various concentrations of protein kinase A. FIG. 4B is a plot of RFU versus time at various concentrations of inhibitor at an enzyme concentration of 20 nM.

FIG. 5 is a plot of delta RFU versus ATP concentration in a phosphosinositide-3-kinase a assay showing ATP tolerance curve for the assay.

FIG. 6 is a plot of velocity versus substrate concentration for the kinase PKA.

FIG. 7A is a plot of RFU (perpendicular) versus % phosphoprotein at various fluorescein-labeled phosphoprotein concentrations.

FIG. 7B is a plot of RFU (parallel) versus % phosphoprotein at various fluorescein-labeled phosphoprotein concentrations.

FIG. 7C is a plot of the ratio of RFU perpendicular to RFU parallel showing simultaneous monitoring of fluorescence quench and polarization of dye-labeled substrate as a function of phosphorylation.

FIG. 8 is a bar graph showing delta FP of sensor with increased concentration of zirconium ion.

FIG. 9 is a plot of RFU versus wavelength demonstrating transferred fluorescence emission of a dye-labeled protein upon binding to sensor. The fluorescence energy is transferred from the sensor to the Dylight 647 fluorophore and then emitted.

FIG. 10 is a schematic depicting use of sensor for detection of enzymatic activities other that kinase/phosphatase activity, for example, protease, methylase and/or acetylase activities.

FIG. 11 is a representative synthesis scheme used to generate phosphonate sensor.

FIG. 12 shows simultaneous monitoring of fluorescence quench of substrates labeled with Hylyte488, TAMRA and Chromeo642.

FIG. 13 is a bar graph showing specific detection of activity of a phosphatase in the presence of several substrates.

FIG. 14 is a plot of RFU versus concentration of PKA for Chromeo642-labeled peptide compared to TAMRA labeled peptide.

FIG. 15A is a graph showing % activity as a function of time for a reaction containing Chromeo642-labeled peptide±0.5 nM PKA and ±ATP. FIG. 15B is a graph showing % activity as a function of time for a reaction containing TAMRA-labeled peptide and zero, 3 nM or 6 nM PKA±ATP.

FIG. 16A is a bar graph showing RFU as a function of μg of cell lysate added ±ATP. FIG. 16B is a bar graph showing delta RFU between reactions containing ATP and those not containing ATP as a function of μg lysate added.

FIG. 17A is a bar graph showing RFU at various concentration of ATP in the presence (solid bar) and absence (striped bar) of 25 μg lysate. FIG. 17B is a line graph showing signal to background ratio (S/B) as a function of the ATP concentration in the reaction mixture.

FIG. 18A is a line graph showing the change in relative fluorescence observed as a function of inhibitor concentration for the inhibitors 5-24 (squares) and staurosporine (filled circle). FIG. 18B is a bar graph showing RFU as a function of inhibitor concentration with the inhibitors LR294002 and PI3K I-2±ATP.

FIG. 19 shows the results of biochemical assays for an enzyme dose response curve of phosphodiesterase 4A1A. FIG. 19A shows the results obtained using 2 μM fluorescein labeled cAMP substrate. FIG. 19B shows the determination of Z'factor using PDE4A1A concentrations of 1 nM, 0.25 nM and 0 nM and 2 μM fluorescein labeled cAMP substrate. FIG. 19C shows the results of a kinetic dose response analysis using various enzyme concentrations and 2 μM fluorescein labeled cAMP substrate. Sensor (zirconyl chloride) was used at 100 μM.

FIG. 20 shows a Michaelis-Menten fit of slopes derived from a kinetic experiment in which various concentrations of fluorescein-labeled substrate were incubated with 2 μM fluorescein labeled cAMP substrate, 1 nM enzyme, and 100 μM sensor (zirconyl chloride).

FIG. 21 shows line curves of inhibition biochemical assays for PDE4A1A (3 nM) with various concentrations of the inhibitor RO20-1724 (filled circles) or IBMX (open diamonds) in endpoint (FIG. 21A) or kinetic (FIG. 21B) modes. Assays were conducted with fluorescein-labeled cAMP at 2 μM and sensor (zirconyl chloride) at 100 μM.

FIG. 22 shows cleavage of fluorescein-labeled cAMP as a function of various concentrations of lysates using kinetic monitoring. Assays were conducted with fluorescein-labeled cAMP at 2 μM and sensor (zirconyl chloride) at 100 μM.

FIG. 23 is a bar graph showing simultaneous monitoring of quench of fluorescein-labeled cAMP at 1.5 μM (left axis) and TAMRA-labeled cGMP at (right axis) as a function of the concentration of mouse brain lysate.

FIG. 24A is a schematic that explains the experimental data shown in FIG. 24B. In the presence of cAMP, PKA activity is stimulated resulting in phosphorylation of labeled kemptide substrate and an increase in the delta RFU of the kemptide (right axis). PDE4A 1A hydrolyzes c-AMP to AMP resulting in an increase in the delta RFU (left axis). In the presence of the PDE inhibitor IBMX, hydrolysis of cAMP is inhibited resulting in hight concentrations of cAMP and higher PKA activities. FIG. 24B is a line graph of the change in relative fluorescence units (delta RFU) in fluorescien-labelled cAMP (1.5 μM, left axis) and Chromeo642-labeled LRRASLG (3 μM, right axis) caused by PKA present in 2 μg lysate in the presence of varying amounts of the non-selective PDE inhibitor IBMX. PKA activity increases with increasing amount of IBMX (filled squares) and PDE activity decreased with increasing amounts of IBMX (empty circles).

### DETAILED DESCRIPTION OF THE INVENTION

Diseases such as cancer, diabetes and infection are the result of an inappropriate cellular response to extracellular or intracellular cues, which elicit a specific phenotypic response, such as proliferation or apoptosis. These cellular cues are mediated by a complex interconnected network of bio-molecules that are commonly activated or deactivated by PTM, for example, phosphorylaton. To enhance drug discovery an approach is needed that provides information on signaling networks as a whole rather than simply on one or two components. The present invention provides a multiplex technique that can quantitatively measure variations of multiple protein activities within one or across several signaling pathways. The assays of the invention provide techniques that are both sensitive and fast. The present invention makes use of the fact that a sensor based on electron transfer does not require spectral overlap between donor and acceptor molecules, and one of this type would be ideally suited for multiplexing applications.

Sensors

The present invention provides a novel sensor that may be used to detect and/or quantify the presence of one or more enzymatic activities in a sample.

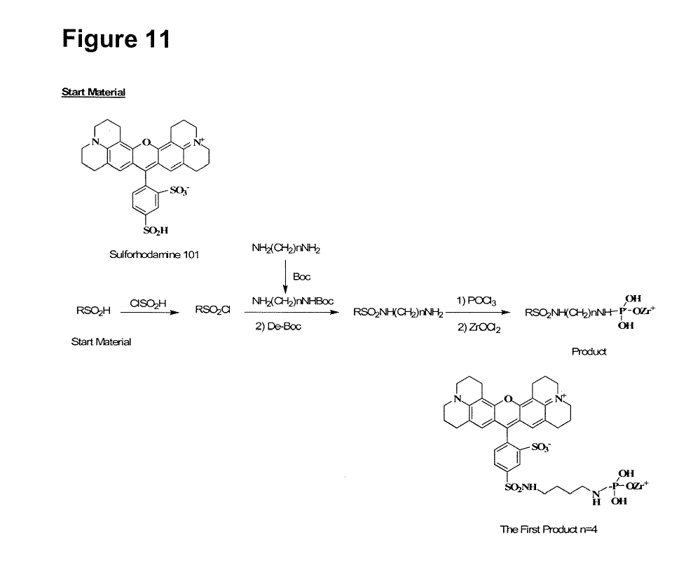

In one embodiment, a sensor of the invention may comprise a fluorescent chelator and a metal ion associated to the chelator. Suitable metal ions include, but are not limited to, zirconium ions. The chelator may be a phosphonated fluorescent molecule that can be generated as described in FIG. 11, or may be purchased commercially from, for example, Active Motif, San Diego, Calif. Examples of phosphonate fluorescent dyes that may be used in this embodiment include, but are not limited to, Chromeo 547 or Chromeo 642 fluors. The structures of these dye fluors are provided in the following structures.

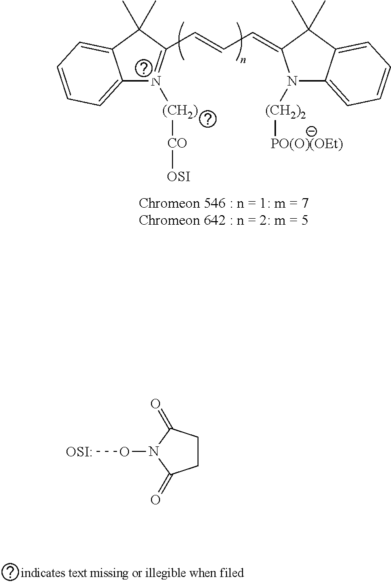

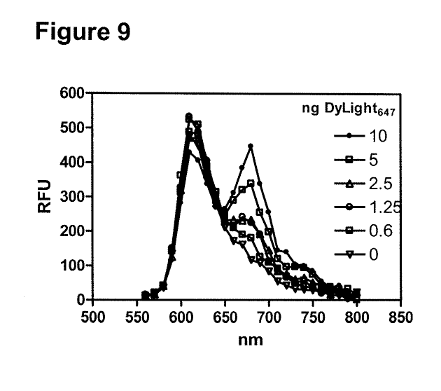

Embodiments of this type (i.e., bi-molecular sensors) are useful for assays in which fluorescence can be transferred from the sensor fluor to an acceptor fluor (see FIG. 9). In addition, sensors of this type may be immobilized on solid surfaces such as glass or plastic via covalent tether or using avidin coated surfaces in conjunction with biotinylated Chromeo642 or Chromeo547, or using streptavidin labeled Chromeo642 or Chromeo547 in conjunction with biotinylated surfaces.

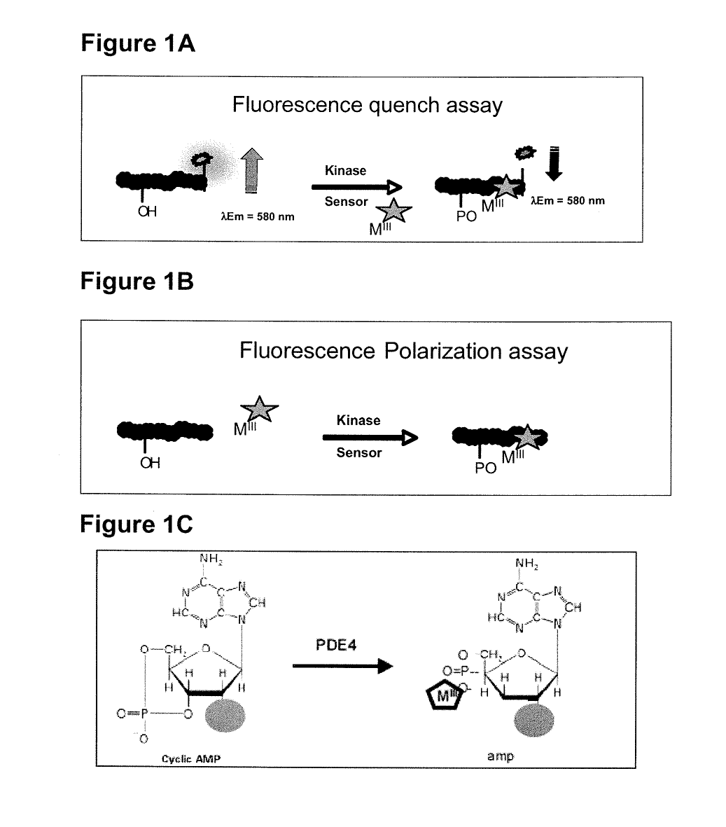

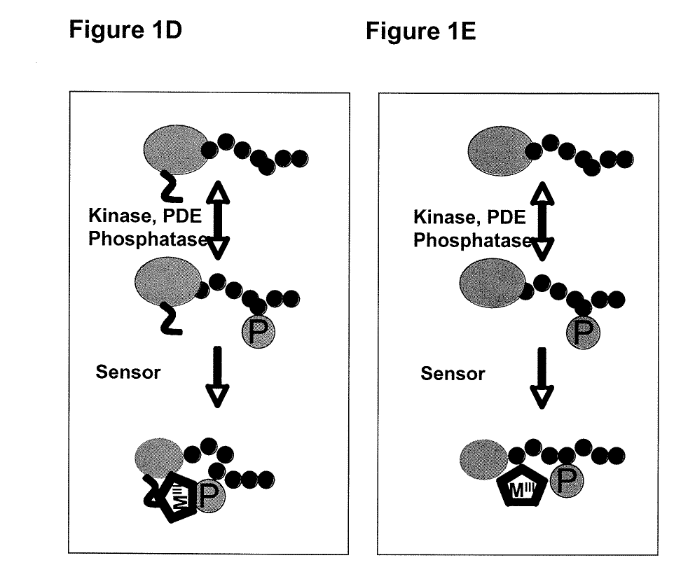

In another embodiment, a sensor of the invention may consist solely of a metal ion (e.g., a zirconium ion). The metal ion may be provided as a suitable salt. In one embodiment, a sensor of the invention may be zirconium which may be supplied as zirconyl chloride. The metal ion can associate with phosphates on a substrate body and quench fluorescence via intermolecular quenching (FIG. 1E) or it can form a ternary complex with phosphonates on fluors (eg Chromeo642 or chromeo547) and phosphates on the body of the substrate (FIG. 1D). The sensors of the invention provide numerous advantages over sensors of the prior art including low cost of materials, high sensitivity, reproducibility, long shelf-life, and the ability to associate with any substrates such as proteins. When proteins such as an antibody are labeled with zirconyl chloride, the antibody sensor can readily quench the fluorescence of a fluorescent target molecule upon specific binding. This allows easy generation of a binding sensor via association of zirconyl chloride to phosphate groups present in the antibody protein.

In one embodiment, the present invention provides a unique sensor that may be used to detect kinase and phosphatase activities by metal ion association to phosphoryl groups. When a fluor-labeled substrate is phosphorylated by a kinase, association of the sensor to the phosphate group brings the fluor and the sensor into proximity allowing electron transfer to occur. Sensors of the invention may operate by inter and/or intra-molecular mechanisms (FIGS, 1E and 1D, respectively).

As shown below, peptide substrates labeled with Fluorescein, TAMRA and Chromeo642 can be quenched simultaneously with this sensor in the presence of cellular lysates. In some embodiments, the metal ion may be associated with a chelator, such as a phosphonate. In some embodiments, the chelator may comprise a bi-phosphonated molecule.

Substrates

Substrates for use in the assays of the invention typically comprise a body portion, a fluorescent moiety, and optionally, a phosphoryl moiety.

The body portion of the substrates of the invention is limited only by the enzyme to be assayed. The body portion will typically be the same as or mimic the naturally occurring substrate of the enzyme of interest. A body portion may be of any chemical make up, for example, may be a peptide or protein, a lipid, a carbohydrate, a nucleic acid etc. Any molecule that is acted upon by an enzyme and to which a fluorescent moiety may be attached can be used as the body portion of a substrate of the invention.

For assays involving enzymes that add a phosphoryl group (e.g., kinases), the body portion of substrate may comprise a hydroxyl group to which a phosphoryl group may be transferred. When assaying a protein kinase, the substrate may comprise an amino acid comprising a hydroxyl group to which a kinase can transfer a phosphoryl group (e.g., a serine and/or tyrosine).

For assays involving the removal of a phosphoryl group (e.g., phosphatase reactions) a substrate of the invention will typically comprise a phosphoryl group attached to the body portion of the substrate.

For assays involving cleavage reactions (e.g., phosphodiesterases, proteases, lipases, etc), the body portion of the substrates of the invention will typically comprise a cleavage site for the enzyme of interest. As a general rule, the cleavage site will be positioned such that after being acted upon by the enzyme, the body portion will be cleaved into two fragments, one of which may comprise a phosphoryl moiety and one of which may comprise the fluorescent moiety. Thus action of the enzyme of interest results in separation of the phosphoryl moiety from the fluorescent moiety and this separation results in a detectable modulation of the fluorescent properties of the substrate (i.e., change in RFU and/or FP). In one embodiment, a phosphodiesterase may be assayed using the sensors of the invention. After a cyclic nucleotide is acted upon by a phosphodiesterase, a phosphate group is produced that can then interact with a sensor of the invention.

Examples of protease cleavage sites that may be incorporated into the substrates of the invention include, but are not limited to, a cleavage site for aminopeptidase M (e.g., amino terminal L amino acids, a cleavage site for carboxypeptidase A which cleaves carboxy-terminal L-amino acids, a cleavage site for cathepsin C which cleaves amino terminal dipeptides, a cleavage site for chymotrypsin which cleaves after F, T or Y; a cleavage site for collagenase which cleaves peptide containing the sequence P—X-G-P after X where X is any amino acid; a cleavage site for endoproteinase Arg-C whcich cleaves peptides containing R—X after R where X is any amino acid, a cleavage site for endoproteinase Asp-N which cleaves peptides containing D and cystic acid before the D or cystic acid, endoproteinase Lys-C which cleaves peptides containing K after the K; a cleavage site for enterokinase, which cleaves peptides containing D-D-D-D-K after the K, a cleavage site for Factor Xa which cleaves peptides containing R after the R, a cleavage site for kallikrein, which cleaves peptides containing R after some R, a cleavage site for plasmin, which cleaves peptides containing K or R after the K or R, and a cleavage site for thrombin which cleaves peptides containing R after the R.

Any suitable fluorescent moiety known to those skilled in the art may be used in the practice of the invention. Suitable fluorescent moieties include, but are not limited to, TAMRA dyes, BODIPY dyes, fluorescein, CHROMEO dyes, DyLight dyes, cyanine dyes, R-phycoerythrin (PE), fluorescein, lissamine rhodamine B, Texas Red, allophycocyanin (APC), Cy3.5, Cy 5.5, and Cy7.

Assays

In general, the methods of the present invention involve assaying the activity of an enzyme of interest by contacting the enzyme with a population of fluorophore labeled substrate in an aqueous enzymatic reaction mixture and allowing the enzymatic reaction to proceed for a desired period of time and temperature. The reaction is then brought into contact with a sensor, which may be fluorescent or non-fluorescent. A sensor of the invention may comprise or be associated with zirconyl chloride and may form a complex with the phopshoiylated substrate. This complex of sensor and substrate results in fluorescence modulation of the fluorophore labeled substrate. Complexes formed as described above can be detected using unlabeled substrates by monitoring alterations of FP of the sensor. By measuring the change of the observed intensity of fluorescence or fluorescence polarization from the mixture and relating the same to that of a reference, a differential signal can be identified and quantified. The amount of change of fluorescent signal of the sample in indicative of the final state of the fluorophore labeled substrate population, and, in turn, reflects enzymatic activity.

The methods of this invention are applicable for the assay of kinase, phosphatase, and/or phosphodiesterase activities using peptide, proteins, lipids, carbohydrates, and/or nucleic acids (e.g., cyclic nucleotides) as substrates. The enzyme is reacted with substrate to produce an end product containing a phosphoryl group having binding affinity for the metal ion. In one embodiment the substrate contains an attached fluorophore label and its fluorescence quench is monitored following conclusion of the enzymatic reaction upon addition of a sensor to the reaction mix. A further embodiment demonstrates the linearity of signal that can be obtained using substrates labeled with different fluorophores such as Fluorescein, Hilyte488, TAMRA or BODIPY-TMR. Another embodiment describes the kinetic monitoring that can be accomplished by adding fluorescent sensor to the reaction mix and detecting fluorescence quench as it occurs in real time. Another embodiment demonstrates the ability of simultaneous monitoring of the distinct emissions of several fluorophores with the sensor in defined medium or in complex cellular lysates. In another embodiment the change in FP of a fluorescent-labeled substrate is quantified. Another embodiment demonstrates the ability of monitoring sensitized emission from the sensor to a fluorophore labeled protein. An additional embodiment demonstrates the monitoring of measuring changes in FP of the sensor in the presence of unlabeled peptide substrate.

An assay of the invention may configured in a variety of ways. For example, a kinase assay can result in either a decrease (fluorescence quench) or an increase (FP or transferred emission) in the detected fluorescent signal. The signal changes are proportional to enzyme activity and result in linear dose responses. In a particular embodiment, a substrate comprises a fluorescent moiety comprising a phosphonate moiety. The substrate may also comprise a phosphate moiety positioned such that, upon addition of a metal ion (e.g., a zirconium ion) both the phosphonate and the phosphate are coordinated to the metal ion. The phosphate may be attached to the substrate before the reaction (for example, in a phosphatase assay) or as a result of the reaction (for example, in a kinase assay) so that addition or removal of the phosphate group can be monitored by the changes in fluorescence quenching observed.

Increased sensitivity can be obtained using fluorophores that are labeled with a phosphonate by virtue of providing a bridge between the phosphonate-sensor-phosphate that brings the sensor into closer proximity to the fluorophore, resulting in enhanced quench.

With respect to phosphatase assays, in fluorescence quench assays, the initial starting enzymatic reaction will be more highly quenched prior to reaction by virtue of the fluorophore labeled substrate being already phosphorylated. Thus the observed fluorescence emission from the population after enzymatic removal of the phosphoryl group will increase.

Protease and/or lipase activity can also be measured by the method of the present invention. In this case a substrate is selected to have a cleavage site between the attached fluorophore and the phosphoryl group. Upon cleavage with a lipase or protease, the sensor is added and allowed to associate to the phosphate. In the absence of cleavage, fluorescence is quenched due to the proximity of the substrate fluorophore with the sensor, whereas in the presence of cleavage, the fluorescent species are removed from another to an extent that disrupts energy transfer.

Another aspect of the invention is making use of phosphoryl groups as a biological tag with which fluorescent sensor can bind via metal-ion association. In this manner the presence of other post-translationally active enzymes, such as methylases and transferases can be monitored. In this case, a substrate is selected which is labeled with a fluorophore at one site and with a phosphoryl group at another site. The sequence of the substrate is such that it contains a recognition site for a specific post-translationally active enzyme that is also recognized by a cleavage enzyme (e.g., a protease or a lipase). In the presence of activity of the post-translationally active enzyme, a chemical group such as a methyl or acetyl group is transferred to the substrate, which interferes with the ability of substrate to be cleaved by a secondary cleavage enzyme. Upon addition of sensor to the substrate, a modulation in fluorescence can be observed as described above.

The binding or disassociation of two proteins can be detected by monitoring modulation of FP of one of two proteins tagged with sensor via metal ion/phosphate interaction following binding or disassociation with another protein.

A further embodiment of the invention is a synthesis scheme designed to produce a composition comprising a fluorophore with a phosphoryl group as a receptor to which a metal ion can bind. The complex retains its ability to associate with phosphoryl groups present on biological substrates and causes modulation of fluorescence of the substrate label when a complex is formed. Suitable fluorophores span the range of the visible spectrum (˜400 nm-750 nm) and are able to act as either donor or acceptor fluorophores to a fluorophore labeled substrates via energy or electron transfer mechanism. Another embodiment of the invention describes the preparation of zirconyl chloride complexes that are capable of associating with phosphates present on fluorophore labeled substrates or to phosphonate groups present on the fluorophore.

Yet a further embodiment of the inventions provides a kit comprised of one or more of a metal ion (e.g., Zr⁺⁴), a peptide, a peptide labeled with a phosphonate-modified fluorophore (e.g., a Chromeo fluorophore), association buffer, postreaction buffers, sensor dilution buffers and reaction buffers appropriate for an enzyme of interest as well as an instruction booklet describing the manner in which the assay can be accomplished with respect to one or more enzymes. The kit may include a synthetically prepared calibrator to function as external reference. The calibrator may comprise a synthetic substrate labeled with a phosphoryl group. Labeled substrates (e.g., peptides) may be provided in kits of the invention or may be supplied by the user. Substrates may be labeled using any suitable label including, but not limited to, fluorescein and its derivatives, TAMRA and its derivatives, Cy5 and its derivatives or any fluorescent molecule spanning the UV-visible range.

The present invention provides assays that can measure the phosphorylation of any substrate by any kinase with one universal approach. Such generic assays include those based on metal ion chelates that can directly associate to phosphorylated proteins and peptides. Without wishing to be bound by theory, the present invention makes use of electron transfer quenching of a fluor-labeled substrate by a metal ion (FIG. 1). The metal ion may be used alone or may be coordinated with a chelator, for example, a phosphonate such as those is present on Chromeo fluors to form a stable complex. The complex retains its ability to associate to phosphoryl groups present on serine, threonine or tyrosine amino acids on peptide substrates, to phosphorylated lipids or to DNA substrates. Upon association of the complex to the phosphoryl groups of a dye-labeled substrate, the metal ion is brought into a proximity that allows electron transfer to occur. As a result, the fluorescence intensity of the substrate decreases proportionally to the increased percentage of phosphorylation.

The fluorescence of a fluorophore label on an enzymatic substrate can be altered by the presence of a sensor of the invention when brought into proximity to the substrate fluorophore, for example, via metal-ion and phosphate interaction. The change in fluorescence of the substrate can be monitored as fluorescent quench, transferred emission or change in FP. Alternatively, modulation of FP as well as fluorescence quench of a fluorophore labeled substrate can be measured simultaneously whilst monitoring the parallel and perpendicular emission required for FP. Additionally, changes in FP of the sensor can be measured in the presence of unlabeled substrate. Additionally, simultaneous modulation of fluorescence quench of substrates labeled with various fluorophores can be measured in multiplexed mode. Finally, measurement can be made in defined assay conditions as well as in the presence of cellular lysates, enabling the dissection of signaling pathway events within lysates of cells.

In some embodiments, activity assays of the invention quantify phosphorylation of a synthetic substrate by an activated kinase. In some embodiments, the present invention may provide assays of enhanced sensitivity. Such assays may comprise conjugation of the chelating fluors directly to biomoelcules, for example, peptide substrates of kinase and/or phosphatase enzymes. As shown below in the examples, this approach improved the sensitivity of Protein Kinase A (PKA) activity detection 35-fold using Chromeo642 labeled substrates as opposed to substrates labeled with TMR or Fluorescein.

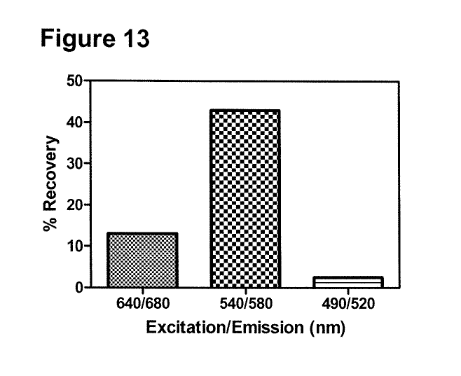

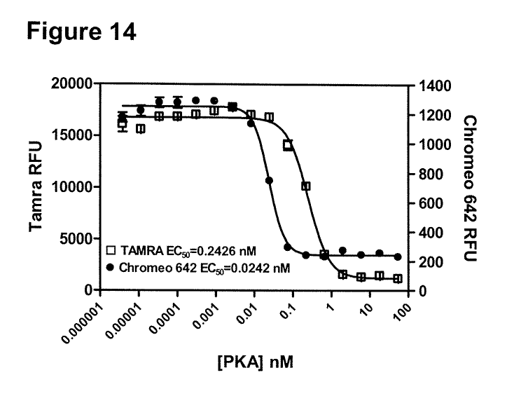

Depending on the location of the chelating group, inter- or intra-molecular quenching is achieved: with the inter-molecular sensor the Chromeo-metal ion complex is assembled and added to a fluor-labeled substrate following incubation with a kinase and ATP. With the intra-molecular approach the metal ion co-ordinating fluor is directly conjugated to the substrate and metal ion coordinated at the end of the reaction. We have shown that intra molecular quenching using a Chromeo642 labeled substrate results in sensitivities of kinase detection that are 35-fold higher than those that can be achieved using peptides labeled with TAMRA or Fluorescein (FIG. 14). In some embodiments, instead of using phosphonated fluors (e.g., Chromeo fluors) as a partner for the formation of an intramolecular ternary complex, the distance between the metal ion and fluor can be reduced by adding a linker that contains a phosphonate group between the fluor and the first (or last) position of the substrate body. Thus, body of the substrate may comprise a phosphonate group which can interact with the metal ion. Embodiments of this type allow any fluor to be used for labeling substrates allowing for larger multiplexed samples. Any of the above embodiments and combinations thereof can be used in multiplex applications to simultaneously detect the presence of multiple enzyme activities in the same sample. We have shown that peptides labeled with either Fluorescein, TMR or Chromeo642 can be quenched individually or simultaneously. The multiplex approach was validated for detection of Protein Phosphatase 1B (PTP-1B) activity in the presence of cellular lysates (FIG. 13).

Multiplex Assays

Fluorescence quench can be accomplished by electron transfer rather than by energy transfer. This involves the physical exchange of an electron from the excited acceptor fluor to the donor fluor. The transfer does not involve a dipole-dipole coupling mechanism as is the case for FRET, and therefore molecules capable of electron transfer do not require spectral overlap. One electron transfer acceptor molecule can therefore potentially quench the fluorescence of any fluor. Thus, a sensor that is capable of electron transfer is ideally suited for multiplexing applications.

Sensors of the invention may be used to conduct homogeneous, multiplexable fluorescent assays. In one example, methods of the invention may be used to simultaneously monitor a plurality (e.g., 2, 3, 4, 5, etc) of enzymes (e.g., kinases and/or phosphatases) involved in one or more signaling pathways. An example of a signaling pathway that can be monitored using the assays of the invention is the phophoinositide kinase 3 (PI3K) pathway, for example, in a human cancer cell line. This profiling of kinase activities within cells has the potential to greatly enhance the diagnosis and treatment of a broad range of human diseases.

To obtain cellular profiles of signaling pathways, the present invention provides a multiplexable, homogeneous kinase activity assay, which has a broad spectrum of application and is adaptable to cellular lysates and high through-put. The present invention may be practiced using commercially available fluorescent moieties, for example, the Chromeo series of fluors from ActiveMotif. The present invention has allowed the development of inter- and intra-molecular sensors and quantification of variations in cellular signaling pathways with higher sensitivity than otherwise possible. In contrast, electron and charge transfer quenching can be accomplished using only one fluorophore, which does require spectral overlap with a donor fluorophore. The ability to quench the emission of a variety of fluorophores enables electron/charge transfer sensors to be used in multiplex application. The use of a non-fluorescent sensor enables the ability to add large concentrations of sensor without generating background fluorescence that can interfere with the assay. In this manner, non-specific binding of the sensor to reagents commonly present in assays such as ATP, EDTA and protein which can cause decease in assay performance can be overcome by addition of higher amounts of sensor. Thus optimized assays afford high tolerance to ATP and substrate, allowing accurate determination of substrate and ATP Km. Additionally, assays are adaptable to large amounts of proteins such as is present in cellular lysates and thus enable monitoring of endogenous enzyme activities. The combined ability to generate highly sensitive assays in cellular lysates in a multiplex fashion enables dissection of signaling pathways networks in response to exogenous stimuli such as administration of chemical compounds that alter the activity of some components of the signaling pathway.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

General Scheme for Assays of the Present Invention.

FIG. 1 provides a schematic representation of one embodiment of the present invention. As shown in FIG. 1, assays of the invention may be quench assays or FP assays.

Typically, assays of the invention may involve 2 steps. In a first step, a fluorescent-dye-labeled substrate (black circles with starburst) is contacted with a sample suspected of having one or more enzymatic activities, e.g.,protease activity, kinase activity, lipase activity, phosphodiesterase activity, and/or phosphatase activity.

In situations where detecting kinase activity is desirable, the substrate is contacted with the sample in a reaction buffer that comprises ATP. The substrate may comprise an amino acid sequence that is specifically recognized by a kinase of interest. The reaction is allowed to proceed for a selected period of time. The substrate may be phosphorylated by the kinase of interest present in the sample. In step 2, a sensor molecule is added to the reaction mixture and incubated for a selected period of time. The sensor associates with the phosphoryl group on the substrate molecules that have been acted on by the kinase of interest. Following association of the sensor (grey star) to the phosphorylated substrate, fluorescence quench of the substrate is monitored. The fluorescence intensity of the substrate decreases proportionally to the increased percentage of phosphorylation. Fluorescence polarization of dye-labeled substrate is detected as increase in fluorescence polarization signal (FIG. 1B).

In situations where detecting phosphatase activity is desirable, the substrate is contacted with the sample in a reaction buffer. The substrate may comprise an amino acid sequence that is specifically recognized by a phosphatase of interest that comprises a phosphorylated residue. The reaction is allowed to proceed for a selected period of time. The substrate may be de-phosphorylated by the phophatase of interest present in the sample. In step 2, a sensor molecule is added to the reaction mixture and incubated for a selected period of time. The sensor associates with the phosphoryl group on the substrate molecules that have not been acted on by the phosphatase of interest. Following association of the sensor (grey star) to the remaining phosphorylated substrate, fluorescence quench of the substrate is monitored. The reduction in quenching (i.e., increase in fluorescence intensity) of the de-phosphorylated substrate is proportional to the increased percentage of de-phosphorylation. Fluorescence polarization of dye-labeled substrate is detected as a decrease in fluorescence polarization signal.

In situations where detecting phosphodiesterase activity is desirable, the substrate is contacted with the sample in a reaction buffer. The substrate may comprise a fluor-labeled cyclic nucleotide (e.g., cAMP or cGMP) or analog thereof that is specifically recognized by a phosphodiesterase of interest (FIG. 1C). The reaction is allowed to proceed for a selected period of time. The reaction of the cyclic nucleotide with the phosphodiesterase results in a nucleotide with a phosphate group that can interact with the sensors of the invention. In step 2, a sensor molecule is added to the reaction mixture and incubated for a selected period of time. The sensor associates with the phosphoryl group on the substrate molecules that have been acted on by the phosphodiesterase of interest.

FIG. 1D is a schematic of intramolecular quenching in which a zirconyl sensor forms a ternary complex between a phosphate on the body portion of a substrate and with a phopshonate on the fluor. In contrast, intermolecular quenching (FIG. 1E) is achieved using a zirconyl sensor or a bimolecular sensor, which associates to phosphates on the body portion of the substrate.

### Example 2

Calibration Curves

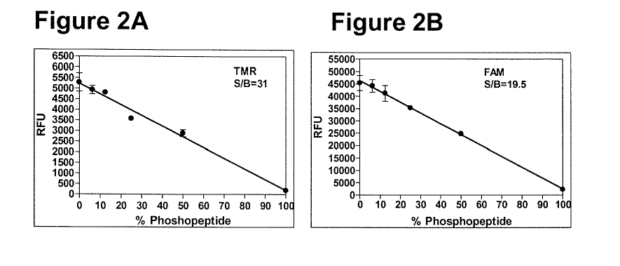

Fluorescent energy transfer from the dye-labeled substrates to the sensor follow a 1:1 ratio, thus resulting in a linear dose response curve. Substrates labeled with Fluorescein, TAMRA and their analogs are suitable substrates for use with the sensors of the invention. When mixtures of phosphorylated and non-phosphorylated peptides are combined in various ratios, a linear calibrator curve is obtained. As shown in FIG. 2, the amount of quenching observed is proportional to the amount of phophorylated peptide present in the sample. TMR- or FAM-labeled, LRRASLG (SEQ ID NO:1) peptide substrates (10 μM) were mixed in various ratios of phosphorylated and non-phosphorylated peptide. Following addition of sensor (100 μM Sulforhodamine 101—Zirconyl sensor), linear calibration curves with Signal to Backgrounds (S/B) of 31 and 19.5 for TMR and FAM, respectively, were obtained.

Sensors of the invention associate with phosphoryl groups on substrates of various chemical natures such as peptide substrates containing phosphoserine, phosphothreonine or phosphotyrosine or phosphorylated lipid substrates. When calibration curves are included in the experimental setup, the precise amount of substrate conversion can be determined.

### Example 3

Enzyme Dose Response Curves

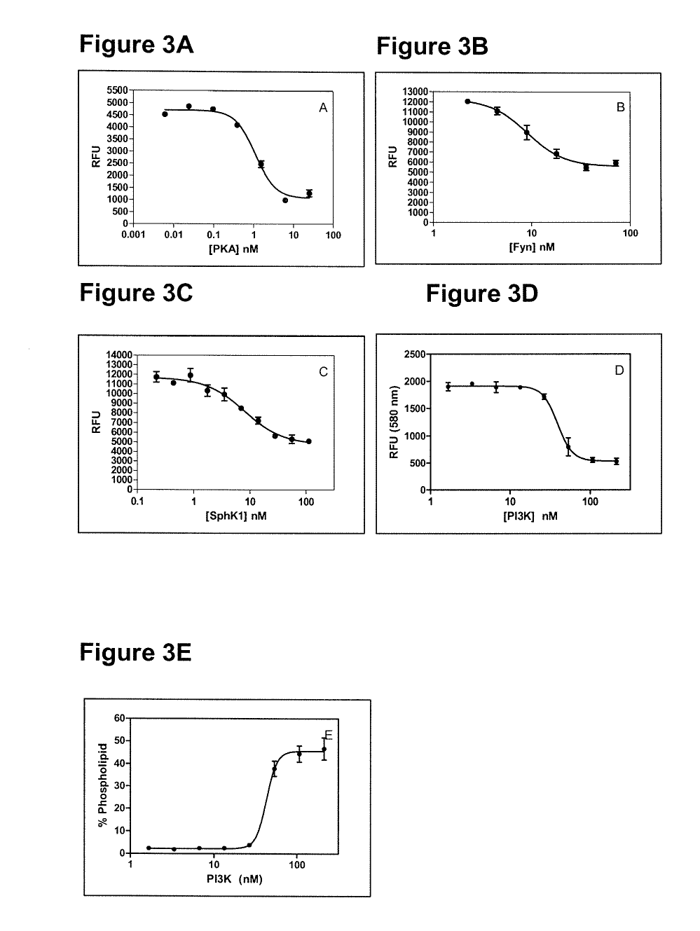

As shown in FIG. 3, assays of the invention can be used to detect the presence and to quantify the amount of enzymatic activity present in a sample. FIG. 3A shows the results obtained using protein kinase a (PKA). PKA was serially diluted in assay buffer (10 mM TRIS, 10 mM MgCl₂, 0.1% BSA, pH 7.2) in the presence of 10 μM ATP and substrate (10 μM HiLyte₄₈₈-LRRASLG). FIG. 3B shows the results obtained with the Fyn, a member of the Src family of kinases. Reaction conditions for Fyn were 50 μM ATP and 10 μM substrate (TAMRA-KVEKIGEGTYGVVYK) in assay buffer (10 mM TRIS, 10 mM MgCl₂, 0.1% BSA, pH 7.2). As shown in FIG. 3C, Sphingosine Kinase I was reacted with 4 μM TAMRA-Sphingosine in 10 mM TRIS, pH 7.2, 10 mM MgCl₂, 0.01% Triton X-100 buffer in the presence of 50 μM ATP. FIG. 3D shows the results obtained with Phosphoinositide 3-kinase (PI3K). PI3Kα was reacted in 25 mM HEPES, pH 7.4, 50 mM MgCl₂, 5 mM DTT and 50 μM ATP with 1 μM BODIPY-TMR-phosphatidylinositol and the product conversion determined (FIG. 3E) using back calculation with a calibration curve performed simultaneously with the enzyme reaction. Reactions proceeded for 1 hour at room temperature in wells of a 384-well plate followed by addition of 5 μL stop buffer (1M NaCL; 100 mM MgCl₂; 0.015% Brij 35; 0.01% Glycerol; 0.05% NaN₃) and 30 μL sensor (100 μM Sulforhodamine 101—Zirconyl sensor) diluted 1:20 in sensor dilution buffer (10 mM MES, pH 6.5; 1M NaCl; 0.01% Triton X-100; 0.05% NaN₃). Fluorescence quench was monitored after 1 hour with an excitation wavelength of 450 or 540 nm and 490 or 590 nm emission for HiLyte₄₈₈ or BODIPY-TAMRA, respectively. Curve fit was performed using sigmoidal dose response.

### Example 4

Real Time Assays

The monitoring of enzyme activity as it occurs in “real time” simplifies the optimization of assay parameters, the establishment of Michaelis-Menten constants for substrate and ATP well as the determination of the mode of action of inhibitors. In contrast to other detection platforms (e.g., other metal ion-phosphate-based systems), the sensors used in this invention associate with phosphate at physiological pH, thus allowing monitoring of actual enzyme activity as it occurs.

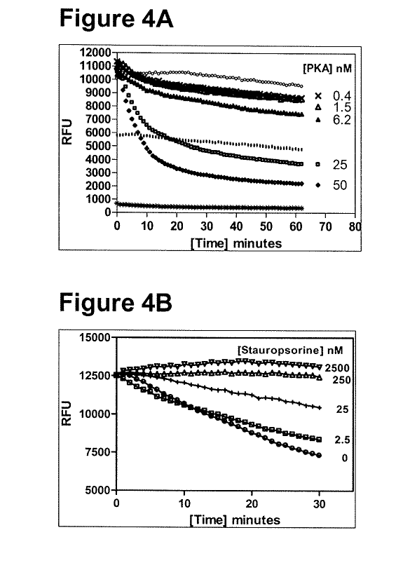

The results of a real time assay are shown in FIG. 4. Various dilutions of PKA in assay buffer and 50 μM ATP were added to wells of a 384-well plate in the presence of 250 nM sensor (FIG. 4A). Dilutions of Staurosporine were added to assay buffer containing 50 μM ATP in the presence of 250 nM sensor (FIG. 4B). Fluorescence quench was monitored at 450 nm excitation and 490 nm emission in 1-minute intervals.

### Example 5

Effects of ATP on Assay Sensitivity

The sensor tolerates concentrations of ATP up to 1 mM with minimal loss of signal. This allows to establish relevant ATP Kₘ and further, the screening of structurally diverse libraries for non-ATP competitive inhibitors, which requires high ATP tolerance of the screening platform.

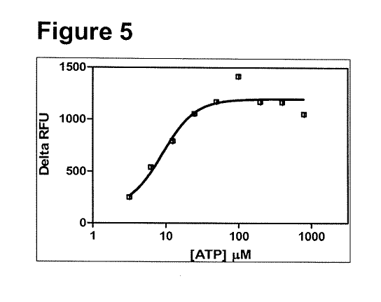

FIG. 5 shows an ATP tolerance curve for PI3Kα. BODIPY-TMR Phosphatidylinositol (1 μM) was added to various concentrations of ATP in the presence or absence of 44 nM PI3Kα and the reaction stopped by addition of stop buffer. Sensor was added and the delta RFU between reactions with and without enzyme calculated.

### Example 6

Effects of Substrate Concentration on Assay Sensitivity

In contrast to most fluorescence-based platforms, the sensor tolerates high concentrations of substrate. Substrate concentrations can vary between 100 nM to 200 μM, which allows establishing relevant Kₘ and identification of substrate-competitive inhibitors in the presence of high concentrations of substrate.

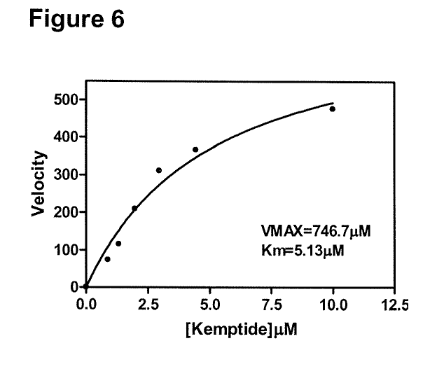

FIG. 6 shows a Michaelis-Menten Plot for substrate. PKA (12 nM) and sensor were added to various concentrations of Hilyte₄₈₈-labeled Kemptide (a consensus sequence substrate for PKA having the amino acid sequence LRRASLG) and substrate conversion monitored in kinetic mode using 450 nm excitation and 490 nm emission. Slopes were plotted against the concentration of substrate and Vₘₐₓ and Kₘ calculated using Michaelis-Menten equation in GraphPad Prism.

### Example 7

Fluorescence Polarization (FP) Assays

Upon association of the sensor to phosphorylated substrate, the molecular rotation of the fluorophore-labeled substrate is impeded and polarization of the fluorophore-labeled substrate increased. Since FP measurements are reported as the ratio of the perpendicular and parallel emissions, it is possible to record fluorescence quench seen in the perpendicular and parallel reads simultaneously with FP during one read of a sample well.

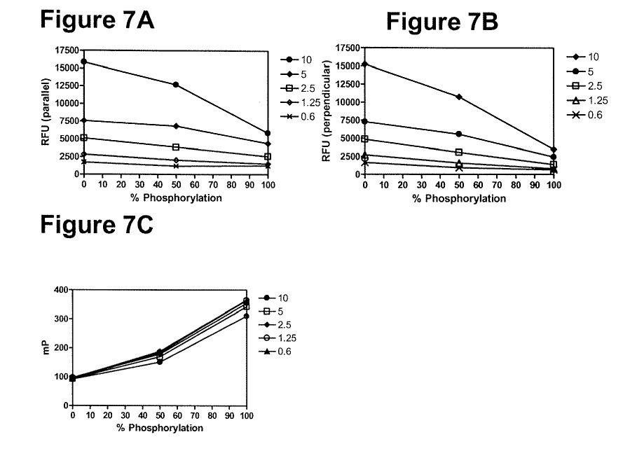

FIG. 7 shows the results of an FP assay. Fluorescein labeled peptides were mixed at the indicated concentrations in reactions containing 20 μM ATP and sensor added. Fluorescence quench was monitored in parallel or perpendicular mode (FIGS. 7A and 7B respectively) and the ratio recorded as fluorescence polarization (FIG. 7C).

### Example 8

FP Assays Using Unlabeled Substrates

The impediment of the molecular rotation of the sensor upon binding to a phosphorylated species can be monitored using unlabeled substrates. In this experiment, we wanted to test the optimal loading of the phosphonate Sulforhodamine 101 chelator with different amount of zirconyl chloride.

Substrate and calibrator peptides were biotinylated LRRASLG (used at 20 μM). 10 μM of the phosphor chelator were incubated for 30 minutes at room temperature with different amounts of metal ion (50 mM). Then the samples were diluted 1:10 in sensor dilution buffer and 30 μL added to 15 μL of 100% phosphor biotin peptide or 0% phosphor biotin peptide. The delta between the samples was plotted.

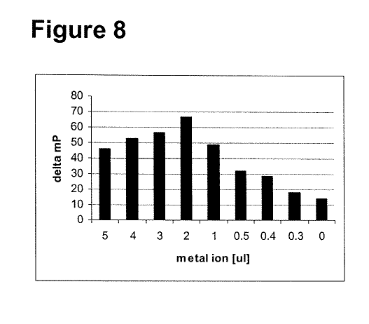

FIG. 8 shows the results of an FP assay using sensor as a readout. Biotinylated phospho and non-phospho peptides were mixed with sensor that had been charged with various dilutions of metal ion. Unlabeled phosphorylated substrate (1 μM) was added to Sensors that were associated to various amounts of Zirconyl chloride (x-axis). The change in fluorescence polarization of the 100 μM Sulforhodamine 101—Zirconyl sensor upon binding to phosphorylated substrate was measured using TAMRA polarization settings. The increase in polarization was determined as the delta between milli pi (mP) of sensor in the absence of metal ion or in the presence of various concentrations of metal ion.

### Example 9

Use of Fluorescence Donors to Excite Other Fluorophores

Energy transfer from the sensor to another fluorescent species results in an emission signal of the acceptor fluorophore upon excitation of the sensor. This results in a “turn on” assay and enables ratiometric monitoring.

A bimolecular type sensor (sulforhodamine 101-phosphonate chelator (FIG. 11)) associated with zirconium ion was used. The complex can associate with phosphates present on streptavidin. The streptavidin was also labeled with DyLight647. When an excitation wavelength of 540 nm is used, the sulforhodamine 101 chelator emits at 590 nm. Since the sensor is associated with the streptavidin-Dylight, the sensor is close enough for energy to transfer from the sulforhodamine 101 to the DyLight fluor. The transferred energy excites the Dylight, which then emits at 685 nm. Transferred emission increases as a function of streptavidin-Dylight647.

FIG. 9 shows the results of an assay using the sensor to excite another fluorescent species. Various ratios of streptavidin labeled with DyLight647 were mixed with sensor and the amount of transferred emission measured in a spectral scan. The height of the peak at 685 nm increases with the amount of added streptavidin DyLight647.

### Example 10

Use of Sensors to Monitor Other Enzymatic Activities

The association of sensor to a phosphorylated site can be used as a tag to monitor activities of other enzymes that are involved in post translational modification (PTM).

In situations where detecting activities of posttranslational enzymes, such as proteases is desirable, the substrate is contacted with the sample in a reaction buffer. The substrate may comprise an amino acid sequence that is specifically cleaved by a protease of interest. In addition, the substrate typically comprises a phosphorylated residue. The cleavage site of the protease may be arranged such that cleavage of the substrate will result in the portion of the substrate comprising the fluorescent label being on a separate fragment of the substrate from the portion of the substrate that comprises the phosphorylated residue. The reaction is allowed to proceed for a selected period of time. In step 2, a sensor molecule is added to the reaction mixture and incubated for a selected period of time. The sensor associates with the substrate fragments comprising the phosphoryl. Following association of the sensor (red star) to the phosphorylated substrate fragments, fluorescence quench of the substrate is monitored. The increase in observed fluorescence intensity is proportional to the increased percentage of cleavage of the substrate. Fluorescence polarization of dye-labeled substrate fragment is detected as a decrease in fluorescence polarization signal.

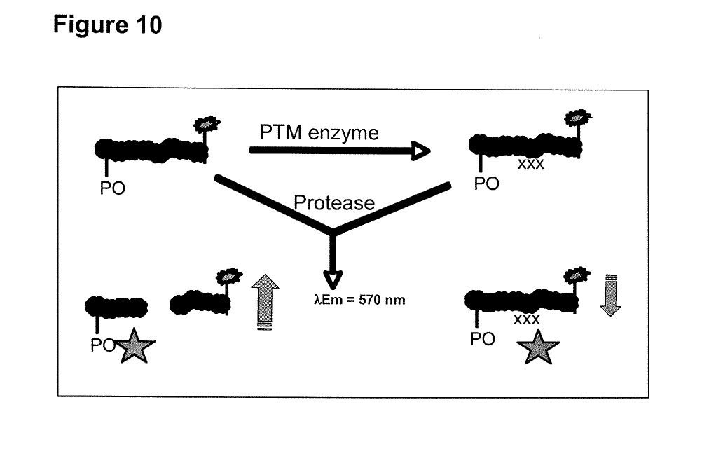

FIG. 10 provides a schematic of assays for detection of PTM activities using a sensor as a tag. A fluorophore-labeled substrate (circles with starburst) is altered by a post-translationally active enzyme. The addition of the modifying group to the substrate (indicated by XXX in FIG. 10) disrupts the ability of a protease to cleave the substrate. The substrate is labeled with a phosphate and can associate with the sensor via metal ion-phosphate interaction. In the presence of cleavage, fluorescence is unquenched, whereas in the absence of cleavage, fluorescence is quenched. Separately, the approach is useful for detecting the activities of proteases in the manner described.

### Example 11

Synthesis of a Sensor of the Invention

FIG. 11 provides a schematic of a synthetic approach for making a sensor of the invention. In the embodiment shown in FIG. 11, the sensor is comprised of a fluorescent chelator and zirconium ion provided as zirconyl chloride.

Sulforhodamine 101 is a precursor for the synthesis. Following amination, the amine group is converted to a phosphonate group using perchloric acid. The metal ion zirconyl chloride is then added and associates with the phosphonate group whilst retaining it's ability to bind to phosphates present on substrates.

### Example 12

Assays Using Multiplex Format

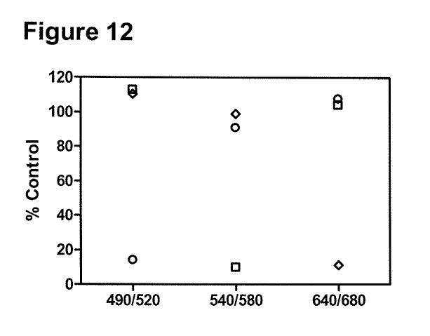

FIG. 12 shows the results of an assay in multiplex format.

Substrates (5 μM LRRASLG) were labeled with Hilyte488, TAMRA or

Chromeo642 and combined into one well containing 5 μM cellular lysates. Sensor was added (30 μL diluted 1:20 in sensor dilution buffer, final concentration 1.25 mM) and fluorescence quench monitored using an excitation wavelength of 490 nm, 540 nm and 642 nm with 520 nm, 590 nm and 680 nm emission for Hilyte488, BODIPY-TAMRA and Chromeo642, respectively

### Example 13

Multiplex Phosphatase Assays

FIG. 13 shows the results from a multiplexed readout of phosphatase activity. Phosphopeptides FAM-LRRA(pS)LG, Chromo₆₄₂-GLRRA(pS)LG and the phosphatase specific TAMRA-GLGF(pY)MAYG were combined into one well and the corresponding non-phosphorylated peptides in another. Following addition of protein tyrosine phosphatase (PTP-1B) sensor was added (30 μL diluted 1:20 in sensor dilution buffer) and fluorescence quench monitored using an excitation wavelength of 490 nm, 540 nm and 642 nm with 520 nm, 590 nm and 680 nm emission for Hilyte₄₈₈, BODIPY-TAMRA and Chromeo642, respectively. The % recovery was calculated based on non phospho controls.

### Example 14

Increased Assay Sensitivity Using Phosphonate Fluors

FIG. 14 shows the increased sensitivity of assays based on intramolecular quenching using phosphonate fluors versus intermolecular quenching. PKA was reacted in assay buffer (10 mM TRIS, 10 mM MgCl₂, 0.01% TritonX-100, pH 7.2) in the presence of 25 μM ATP and substrate (3 μM Chromeo642-GLRRASLG or TAMRA-LRRASLG. Sensor was added (30 μM diluted 1:20 in sensor dilution buffer) and fluorescence quench monitored using an excitation wavelength of 540 nm or 642 nm with 590 nm and 680 nm emission for TAMRA and Chromeo642, respectively. Curve fit was performed using sigmoidal dose response (GraphPad PRISM). The sensitivity of PKA detection using the substrate with phosphonated Chromeo fluor ius 35 times higher than when using TAMRA labeled substrate.

### Example 15

Detection of Enzymatic Activity in Mouse Brain Lysates

The performance of a sensor of the invention for detection of kinase activities in whole mouse brain homogenates was tested using Protein Kinase A (PKA) as an exemplary kinase. A lysis buffer was formulated that effectively terminates protease and phosphatase activities without interfering with the fluorescent signal. With this buffer, lysate was prepared from a total mouse brain and combined with assay buffer, ATP and peptides with sequences specific for PKA (LRRASLG). Peptides were labeled with either TAMRA or Chromeo642, with the expectation that the Chromeo642-labeled peptides would result in higher detection sensitivity.

After incubation at room temperature in 96-well plates, sensor was added and fluorescence quench monitored. Endogenous kemptide phosphorylation was detected in lysates that were incubated with Chromeo642-labeled peptide but not with TAMRA-labeled peptide. It was determined that the majority of kemptide phosphorylation was derived by PKA since the inhibition obtained using the generic serine kinase inhibitor, staurosporine, was almost identical to the inhibition achieved using a PKA-specific competitive substrate inhibitor. Irrelevant inhibitors of Phosphatidylinositol Kinases did not inhibit Kemptide phosphorylation. The sensitivity of the assay was determined to be in the femtomole to attomole range.

Preparation of Mouse Brain Homogenate

Frozen mouse brain (1.5 g) was pulverized using mortar and pestle. Four milliliters of ice cold lysis buffer (Cytobuster Protein Extraction Reagent, EMD Biosciences, San Diego, Calif.) containing a commercially available protease inhibitor cocktail and phosphatase inhibitor cocktails and 1.3 mM DTT. The inhibitor cocktails used were used were 0.12% phosphatase cocktail inhibitor 2 (Sigma Aldrich), 0.5% phosphatase inhibitor cocktail 1 (Sigma Aldrich), ½ complete mini-tablet (Roche), in a buffer containing 20 mM Imidazole; 11.5 mM sodium molybdate and 40 mM sodium tartate. The mixture was transferred to a dounce homogenizer and processed until the preparation appeared homogenous. An additional twelve milliliters of cold lysis buffer was added to the preparation and incubated on a shaker at room temperature for 3 hours. The sample was then centrifuged in a Heraeus Biofuge 13R centrifuge at 13,000 rpm for 20 min after which the supernatant was recovered. Protein concentration was determined by Bichinonic assay (BCA; Pierce) following the manufacturer's recommendations. Concentrated stock aliquots of 1.6 mL and more dilute aliquots of 100 μL were frozen at −20° C. and used fresh for experiments.

Assay

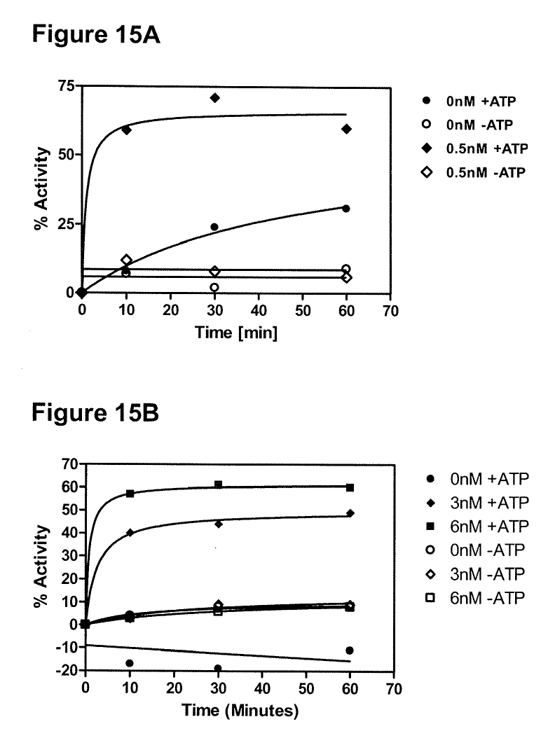

Kemptide (3 μM; LRRASLG) labeled with TAMRA or Chromeo642 was combined with ATP and inhibitor (either staurosporine or PKA inhibitor 5-24, EMD Biosciences, San Diego, Cafif.) in a total reaction volume of 25 μL in wells of a black 96-well plate, which contained 25 μl of lysate. As shown in FIG. 15, the lysate was spiked with varying amounts of PKA and assays were run±PKA and ±ATP. At the end of the incubation period, reactions were terminated by addition of 200 μL of sensor (0.75 mM or 1 mM) diluted in sensor Dilution Buffer (1M NaCl, 10 mM MES pH 6.5; 0.01% Triton X100 0.05% NaN₃). For monitoring of TAMRA fluorescence, excitation and emission wavelengths of 540 nm and 580 nm were used and for monitoring of Chromeo642 fluorescence, wavelengths of 630 nm and 660 nm.

Lysis Buffer Optimization

Phosphorylated and non-phosphorylated TAMRA-labeled peptides were used in the presence of each single lysis buffer component to evaluate possible inhibition with sensor. The Signal to Background (S/B) was determined for each experiment and Sodium orthovanadate found to reduce the S/B substantially (not shown). Therefore, lysis buffer without sodium orthovanadate was prepared using single components. No fluorescence recovery of phosphorylated substrate was observed after 1 hour of incubation at room temperature, indicating effective inhibition of endogenous protease and/or phosphatase activities.

Sensor Optimization

Using kemptide, the concentration of sensor that produced the highest S/B in the presence of 50 μM lysate was determined to be between 0.75 mM-1 mM.

Progress Curves

ATP (25 μM) and either 3 μM Chromeo642-labeled Kemptide or TAMRA-labeled Kemptide were added to 25 μg lysate at various time points. At the end of the 60-minute progress time point reactions were terminated by addition of 1 mM zirconyl chloride as sensor. In order to estimate the amount of endogenous enzyme activities, 6 nM or 3 nM recombinant PKA was added to wells containing TAMRA labeled kemptide (FIG. 15B) and 0.5 nM PKA was added to wells containing Chromeo642-labeled kemptide (FIG. 15A). Following incubation for 1 hour at room temperature reactions were terminated by addition of 100 μM Zirconyl sensor. Control wells were identical to experimental wells but contained no ATP.

The results demonstrate increased sensitivity of the assay using Chromeo-labeled peptides. When 0.5 nM recombinant PKA is spiked into reactions, the reaction is completed after 10 minutes (FIG. 15A), whereas TAMRA-labeled kemptide requires 6 nM PKA for activity to achieve 60% activity (FIG. 15B). FIG. 15 is a plot of % activity relative to the no ATP control.

The progress curves demonstrate endogenous Kemptide phosphorylation in wells containing ATP and no added enzyme (FIG. 15A, “0 nM+ATP”) with Chromeo642 labeled substrate. Following incubation for 1 hour at room temperature reactions were terminated by addition of 100 μM Zirconyl sensor. The assay accurately detects enzyme activity of the 0.5 nM spiked enzyme (FIG. 15A), which corresponds to a sensitivity in the femtomolar range. No endogenous activity was detected with TAMRA labeled substrate (FIG. 15B).

Lysate Optimization

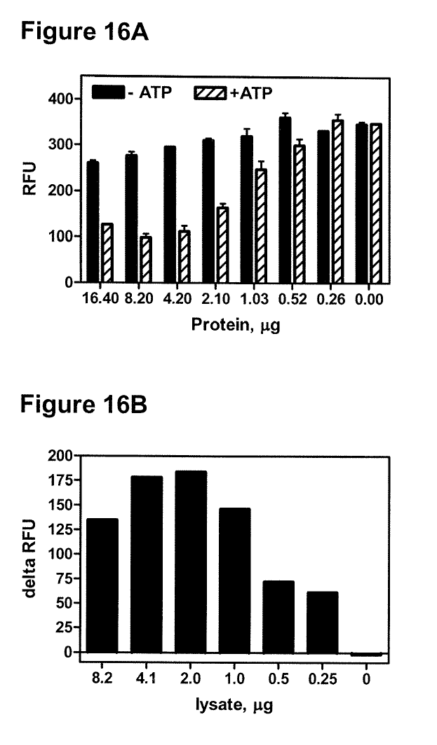

Various concentrations of lysate were tested to determine the smallest amount of lysate that can be used to detect endogenous Chromeo642-Kemptide phosphorylation. Reactions proceeded for 1 hour at room temperature in the absence or presence of ATP (FIG. 16A). Following incubation for 1 hour at room temperature reactions were terminated by addition of 100 μM Zirconyl sensor. The change in relative fluorescence units (delta RFU) was determined (FIG. 16B). Results show that Kemptide phosphorylation can be detected in as little as 0.25 μg lysate.

ATP Optimization

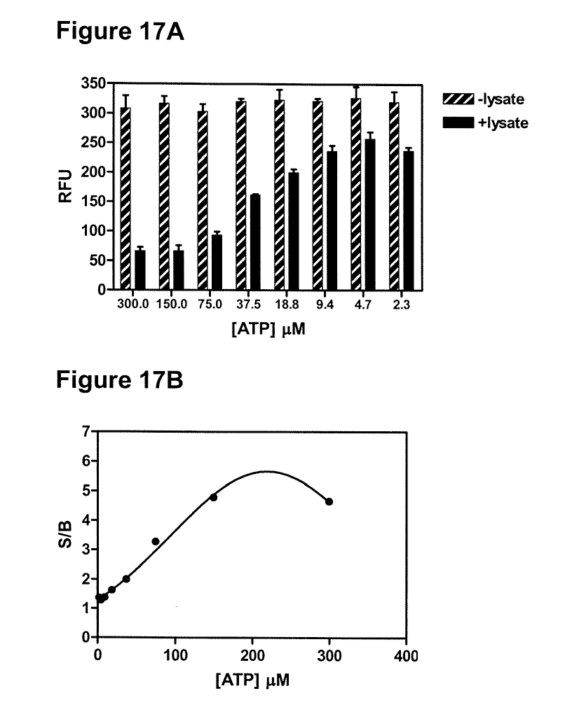

It appears as if endogenous ATP present in the cell is depleted from the lysates during preparation. Therefore, ATP must be added to the reaction to initate kinase activity. To determine the optimal concentration of ATP various concentrations were reacted with Chromeo642 labeled Kemptide (3 μM) in the presence (FIG. 17A, filled bars) or absence (FIG. 17A, striped bars) of 15 μg lysate for 1 hour at room temperature. Reactions were terminated by addition of 100 μM Zirconyl sensor. The S/B was computed (FIG. 17B) and shows that a S/B of >5 can be achieved in the presence of 250 μM ATP.

Inhibition

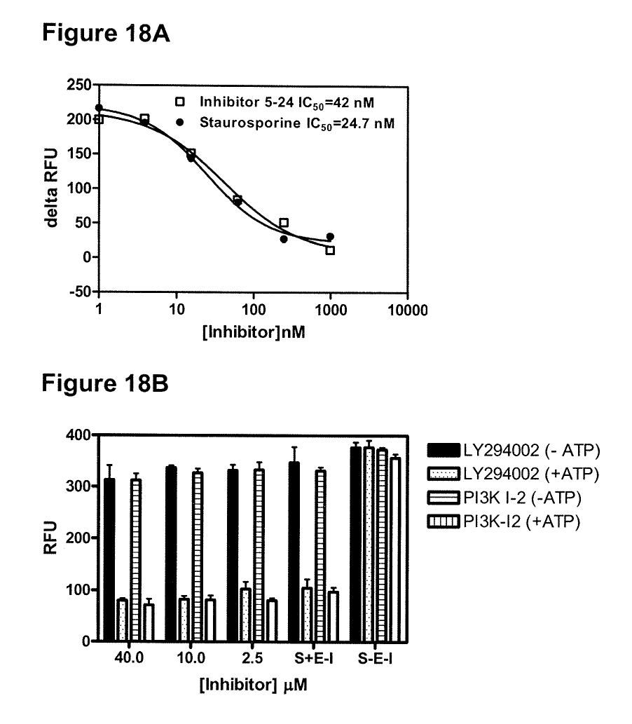

Kemptide (LRRASLG) is a synthetic peptide substrate for cAMP-dependent protein kinase (PKA) derived from the PKA phosphorylation site in liver pyruvate kinase. The substrate is recognized by other kinases, such as members of the PKC family. To evaluate the specificity of Kemptide phosphorylation by PKA, inhibition experiments were performed simultaneously using a generic serine inhibitor, Staurosporine, and a PKA-specific substrate competitive inhibitor (PKA inhibitor 5-24). Reactions were terminated by addition of 100 μM Zirconyl sensor. Staurosporine showed minimally higher potency than the PKA specific inhibitor (FIG. 18A), suggesting that the majority of the Kemptide phosphorylation observed in the mouse brain lysates is derived from PKA. No inhibition was observed using irrelevant inhibitors for PI3K (LY294002 and PI3K I-2; FIG. 18B).

Assays of the invention can be modified in various ways. For example, any source of tissue may be used to prepare a lysate for analysis. In some embodiments, lysates may be prepared from biopsy material taken from a suspected tumor, for example a lung tumor, a breast tumor etc.

In some embodiments, peptide substrates may be designed that are specific for particular kinases. Such peptides may be labeled with a suitable fluorophore, for example, labeled with Chromeo642. Peptides that are specifically phosphorylated by a protein kinase include SEQ ID NOs: 1-12.

In some embodiments, assays of the invention may be multiplex assays (i.e., may be used to simultaneously detect multiple kinase activities) For example, a first peptide substrate labeled with Chromeo642 may be specifically phosphorylated by a first protein kinase and a second peptide substrate labeled with Chromeo546 may be specifically phosphorylated by a second protein kinase. The phosphorylation state of each peptide may be determined as set out above using different excitation and emission wavelengths for each peptide.

### Example 16

Detection of Phosphodiesterase (PDE) Activities.

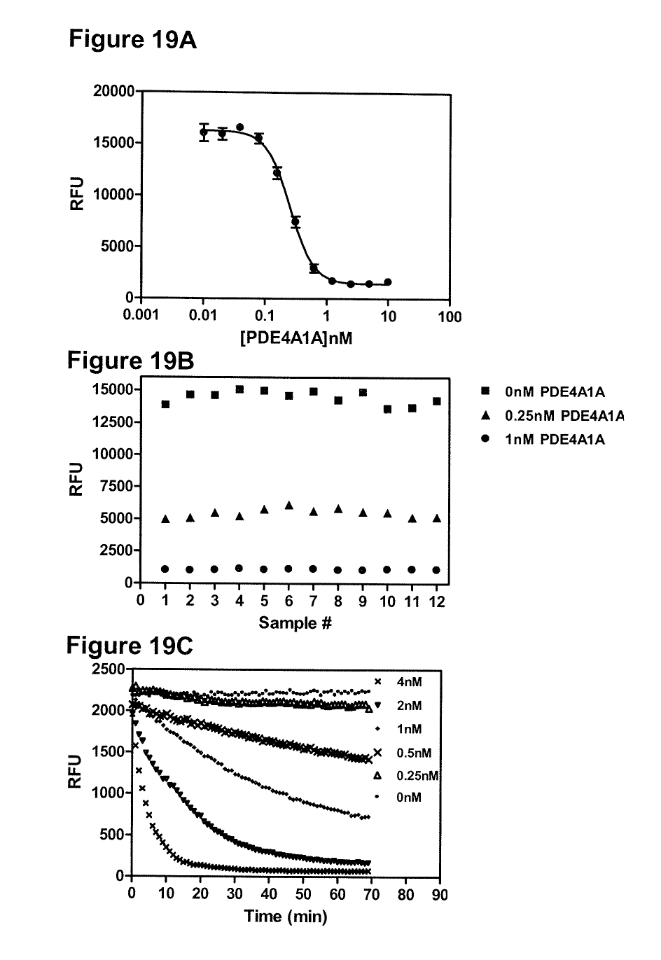

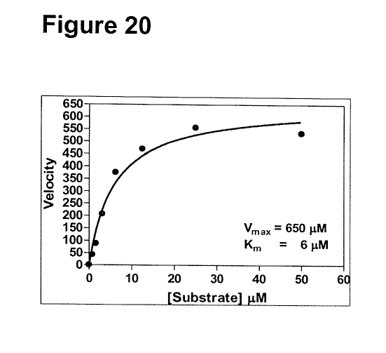

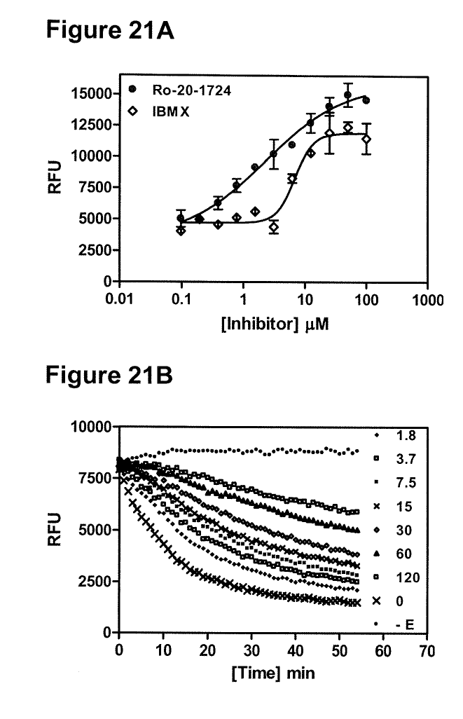

The performance of sensor for detection of phosphodiesterase activities was tested using recombinant PDE41A1A in biochemical assays and using mouse brain homogenates. For biochemical assays, 2 μM fluorescein labeled cAMP was added to various concentrations of recombinant PDE4AIA and zirconyl chloride sensor was added (100 μM) following 1 hour of incubation at room temperature (FIG. 19A). The robustness of the assay was determined by measuring the Z-factor (FIG. 19B). Z-Factors of 0.71 with 1 nM enzyme and 0.88 with 0.25 nM enzyme were achieved. An assay with a Z factor of >0.5 is considered robust enough for high throughput screening. Monitoring of enzyme activities in kinetic mode was achieved using various concentration of enzyme and 2 μM fluorescein labeled cAMP (FIG. 19C). Reactions were terminated by addition of 100 μM Zirconyl sensor. PDE assays tolerate high concentrations of substrate, allowing precise determination of substrate Km (FIG. 20). Reactions were terminated by addition of 100 μM Zirconyl sensor. Inhibition values for the PDE4 specific inhibitor Ro-20-1724 and for the generic inhibitor IBMX closely matched literature values (FIG. 21A). FIG. 21B demonstrates inhibition of PDE41A1A with various concentrations of Ro20-1724 in kinetic mode and in the presence of 100 μM Zirconyl sensor.

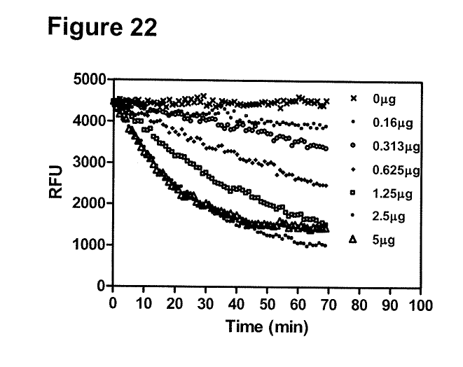

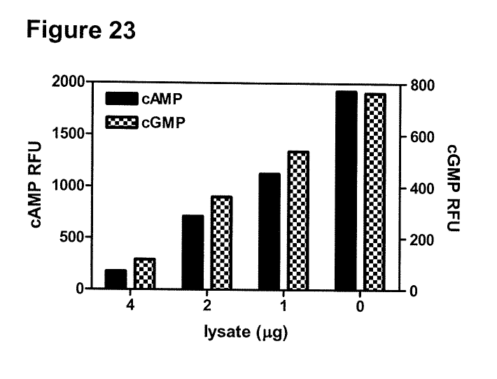

Assays can be performed in lysates of mouse brain, as shown in FIG. 22, using 2 μM fluorescein labeled cAMP and 2 μg lysate and 100 μM zirconyl sensor in kinetic mode. Multiplexing can be accomplished using electron transfer quenching with zirconyl chloride sensor and 2 μM fluorescein labeled cAMP simultaneously with 2 μM TAMRA labeled cGMP in various concentration of mouse brain lysate.

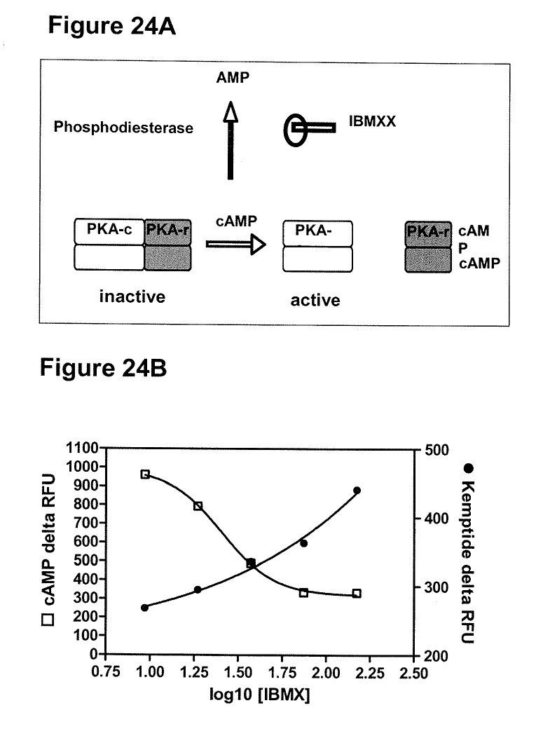

The assay platform can be used to monitor activities of enzymes of various classes, and thus is a novel tool to connect different braches of signaling. As outlined in the schematic FIG. 24A, a phsphodiesterase-mediated cleavage of cAMP is inhibited by IBMX. As a result, increasing amounts of cAMP can bind to the regulatory domain of its downstream target, PKA. Upon binding, the regulatory subunits disassociate from the catalytic subunit and PKA becomes catalytically active. This relationship is experimentally demonstrated in FIG. 24B, which shows increasing phosphorylation of the PKA substrate Chromeo-GLRRASLG and decreasing activity of phosphodiesterase mediated cleavage of cAMP as a function of IBMX concentration.

While the foregoing invention has been described in some detail for purposes of clarity and understanding, it will be appreciated by one skilled in the art from a reading of this disclosure that various changes in form and detail can be made without departing from the true scope of the invention and appended claims. All patents and publications cited herein are entirely incorporated herein by reference.

What is claimed is:

1. A composition comprising a substrate complexed to a metal ion, wherein the substrate comprises:
a fluorescent moiety comprising a phosphonate moiety attached to a body portion;
a phosphoryl group attached to the body portion; and
the metal ion is complexed to the phosphoryl group and the phosphonate group such that at least some fluorescence from the fluorescent moiety is quenched.

2. A composition according to claim 1, wherein the body portion comprises a peptide.

3. A composition according to claim 2, wherein the peptide comprises an amino acid sequence selected from the group consisting of SEQ ID NOs:1-12.

4. A composition according to claim 1, wherein the body comprises a lipid.

5. A composition according to claim 5, wherein the lipid is selected from a group consisting of sphingosine, diacyl glycerol, phosphatidyl-myo-inositol,sphingosine, diacyl glycerol, phosphatidyl-myo-inositol, lipids involved in cellular signaling, phosphatidylinositol phosphates (PIPs), prostaglandins, steroid hormones, estrogen, testosterone, cortisol, oxysterols, and 25-hydroxy-cholesterol.

6. A composition according to claim 1, wherein the body comprises a carbohydrate.

7. A composition according to claim 6, wherein the carbohydrate is selected from the group consisting of myo-inositol, glucose, fructose, and sorbitol.

8. A composition according to claim 1, wherein the fluorescent moiety is selected from a group consisting of TAMRA dyes, BODIPY dyes, fluorescein, CHROMEO dyes, DyLight dyes, cyanine dyes, R-phycoerythrin (PE), fluorescein, lissamine rhodamine B, Texas Red, allophycocyanin (APC), Cy3.5, Cy 5.5, and Cy7.

9. A composition according to claim 1, wherein the metal ion is zirconium.

10. A method of detecting a kinase enzyme in a sample, comprising:
contacting the sample with a substrate for the kinase enzyme, wherein the substrate comprises a fluorescent moiety;
contacting the substrate with a sensor comprising a metal ion; and
detecting fluorescence from the substrate, wherein a decrease in fluorescence indicates the presence of the kinase enzyme.

11. A method according to claim 10, wherein the substrate comprises a body portion.

12. A method according to claim 11, wherein the body portion comprises a peptide.

13. A method according to claim 12, wherein the peptide comprises an amino acid sequence selected from the group consisting of SEQ ID NOs:1-12.

14. A method according to claim 11, wherein the body comprises a lipid.

15. A method according to claim 14, wherein the lipid is selected from a group consisting of sphingosine, diacyl glycerol, phosphatidyl-myo-inositol, lipids involved in cellular signaling, phosphatidylinositol phosphates (PIPs), prostaglandins, steroid hormones, estrogen, testosterone, cortisol, oxysterols, and 25-hydroxy-cholesterol.

16. A method according to claim 11, wherein the body comprises a carbohydrate.

17. A method according to claim 16, wherein the carbohydrate is selected from the group consisting of myo-inositol, glucose, fructose, and sorbitol.

18. A method according to claim 11, wherein the fluorescent moiety is selected from a group consisting of TAMRA dyes, BODIPY dyes, fluorescein, CHROMEO dyes, DyLight dyes, cyanine dyes, R-phycoerythrin (PE), fluorescein, lissamine rhodamine B, Texas Red, allophycocyanin (APC), Cy3.5, Cy 5.5, and Cy7.

19. A method according to claim 11, wherein the metal ion is zirconium.

20. A method of detecting a phosphatase enzyme in a sample, comprising:
contacting the sample with a substrate for the phosphatase enzyme, wherein the substrate comprises a fluorescent moiety and a phosphoryl group;
contacting the substrate with a sensor comprising a metal ion; and
detecting fluorescence from the substrate, wherein an in fluorescence indicates the presence of the phosphatase enzyme.

21. A method according to claim 20, wherein the substrate comprises a body portion.

22. A method according to claim 21, wherein the body portion comprises a peptide.

23. A method according to claim 22, wherein the peptide comprises an amino acid sequence selected from the group consisting of SEQ ID NOs: 1-12.

24. A method according to claim 21, wherein the body comprises a lipid.

25. A method according to claim 24, wherein the lipid is selected from a group consisting of sphingosine, diacyl glycerol, phosphatidyl-myo-inositol, lipids involved in cellular signaling, phosphatidylinositol phosphates (PIPs), prostaglandins, steroid hormones, estrogen, testosterone, cortisol, oxysterols, and 25-hydroxy-cholesterol.

26. A method according to claim 21, wherein the body comprises a carbohydrate.

27. A method according to claim 26, wherein the carbohydrate is selected from the group consisting of myo-inositol, glucose, fructose, and sorbitol.

28. A method according to claim 11, wherein the fluorescent moiety is selected from a group consisting of TAMRA dyes, BODIPY dyes, fluorescein, CHROMEO dyes, DyLight dyes, cyanine dyes, R-phycoerythrin (PE), fluorescein, lissamine rhodamine B, Texas Red, allophycocyanin (APC), Cy3.5, Cy 5.5, and Cy7.

29. A method according to claim 11, wherein the metal ion is zirconium.

30. A method of detecting a protease enzyme in a sample, comprising:
contacting the sample with a substrate for the protease enzyme, wherein the substrate comprises a fluorescent moiety and a phosphoryl group and a peptide sequence recognized by the protease enzyme;
contacting the substrate with a sensor comprising a metal ion; and
detecting fluorescence from the substrate, wherein an increase in fluorescence indicates the presence of the protease enzyme.

31. A method according to claim 30, wherein the peptide comprises an amino acid sequence selected from the group consisting of SEQ ID NOs: 1-12.

32. A method according to claim 30, wherein the fluorescent moiety is selected from a group consisting of TAMRA dyes, BODIPY dyes, fluorescein, CHROMEO dyes, DyLight dyes, cyanine dyes, R-phycoerythrin (PE), fluorescein, lissamine rhodamine B, Texas Red, allophycocyanin (APC), Cy3.5, Cy 5.5, and Cy7.

33. A method according to claim 30, wherein the metal ion is zirconium.

34. A composition comprising a substrate complexed to a metal ion, wherein the substrate comprises:
a fluorescent moiety attached to a body portion;
a phosphoryl group attached to the body portion; and
the metal ion complexed to the phosphoryl group, wherein the phosphoryl group and the fluorescent moiety are positioned on the body such that at least some fluorescence from the fluorescent moiety is quenched.

35. A composition according to claim 34, wherein the body portion comprises a peptide.

36. A composition according to claim 35, wherein the peptide comprises an amino acid sequence selected from the group consisting of SEQ ID NOs:1-12.

37. A composition according to claim 34, wherein the body comprises a lipid.

38. A composition according to claim 37, wherein the lipid is selected from a group consisting of sphingosine, diacyl glycerol, phosphatidyl-myo-inositol, phosphatidylinositol phosphates (PIPs), prostaglandins, steroid hormones, estrogen, testosterone, cortisol, oxysterols, and 25-hydroxy-cholesterol.

39. A composition according to claim 34, wherein the body comprises a carbohydrate.

40. A composition according to claim 39, wherein the carbohydrate is selected from the group consisting of myo-inositol, glucose, fructose, and sorbitol.

41. A composition according to claim 34, wherein the fluorescent moiety is selected from a group consisting of TAMRA dyes, BODIPY dyes, fluorescein, CHROMEO dyes, DyLight dyes, cyanine dyes, R-phycoerythrin (PE), fluorescein, lissamine rhodamine B, Texas Red, allophycocyanin (APC), Cy3.5, Cy 5.5, and Cy7.

42. A composition according to claim 34, wherein the metal ion is zirconium.
